(19) United States
(12) Patent Application Publication
(10) Pub. No.: US 2025/0232578 A1
(43) Pub. Date: Jul. 17, 2025

(54) QUALITY MEASUREMENT OF BASE CALLING IN NEXT GENERATION SEQUENCING

(71) Applicant: Element Biosciences, Inc., San Diego, CA (US)

(72) Inventors: Andrew ALTOMARE, Broomfield, CO (US); Ryan KELLEY, San Diego, CA (US)

(21) Appl. No.: 18/959,159

(22) Filed: Nov. 25, 2024

Related U.S. Application Data
(63) Continuation of application No. PCT/US2023/023605, filed on May 25, 2023.
(60) Provisional application No. 63/346,259, filed on May 26, 2022.

(51) Int. Cl. G06V 10/98; G06F 1/03; G06V 10/70; G06V 20/69

(52) U.S. Cl. CPC G06V 10/993; G06F 1/03; G06V 10/87; G06V 20/69

(57) ABSTRACT

Aspects of the present disclosure relate to a method for predicting quality of base calling in sequencing. A plurality of predictors may be selected and a corresponding value for each of the plurality of predictors may be determined from one or more flow cell images. A quality score may be determined from a look-up table based on the corresponding value for each of the plurality of predictors, wherein the look-up table comprises a plurality of dimensions corresponding to the plurality of predictors and is generated based on a training data set.

### CROSS REFERENCE TO RELATED APPLICATIONS

This application is a continuation of PCT/US2023/023605 filed May 25, 2023, which claims the benefit of U.S. Provisional Patent Application Ser. No. 63/346,259, filed May 26, 2022, which are hereby incorporated by reference in their entireties.

### TECHNICAL FIELD

This disclosure relates generally to determining quality of base calling, and particularly to predicting quality score for performing base-calling in a digital image of a flow cell during DNA sequencing.

### BACKGROUND

Next generation sequencing-by-synthesis, sequencing by binding, or sequencing by avidity using a flow cell may be used for identifying sequences of DNA. As single-stranded DNA fragments from a sequencing library are flooded across a flow cell, the fragments may attach to the surface of the flow cell. An amplification process is then performed on the DNA fragments, such that copies of a given fragment form a cluster or polony of nucleotide strands. In some aspects, a single cluster may attach to the flow cell at random locations.

In next-generation sequencing (NGS) or NGS-like applications such as sequencing by synthesis, sequencing by binding, or sequencing by avidity, in order to identify the sequence of a target nucleic acid, a new strand is synthesized one nucleotide base at a time. During each cycle, 3′-blocked nucleotides attach at complementary positions on the strands, ensuring that only one base will attach to any given strand during a single cycle. In some aspects, such as in sequencing by synthesis, the blocked nucleotide may also be fluorescently labeled, while in others, such as in sequencing by binding or sequencing by avidity, a label is reversibly or noncovalently bound to the synthesis complex in a separate step that takes place after the blocked nucleotide has been incorporated. During the detection step, the flow cell is exposed to excitation light, exciting the labels and causing them to fluoresce. Because, in most existing aspects, the strands undergoing sequencing are clustered together, the fluorescent signal for any one fragment is amplified by the signal from its clonal counterparts, such that the fluorescence for an entire colony may be recorded by an imager. To initiate subsequent sequencing steps, the blocking groups are then cleaved, the surface is washed, and the cycle repeats. Importantly, at the imaging step of each sequencing cycle, one or more images are recorded. A base-calling algorithm is applied to the recorded images to “read” the successive signals from each cluster or polony and convert the optical signals into an identification of the nucleotide base sequence added to each fragment. Errors may be introduced in base calling due to many reasons including the base-calling algorithm and the sequencing steps prior to it. However, it remains a challenge to accurately measure the quality of base calling. Since the measurement is made without requiring knowledge of the sequence to be read, the quality is a predictive measurement of error rate, that is, the base calling from the sequence to be read should have an actual error rate that corresponds to the predictive measurement. A quality score is based on base calling error rate is such a predictive measurement.

### SUMMARY

Provided herein are system, apparatus, method, and/or computer program product aspects, and/or combinations and sub-combinations thereof which enables predictive measurement of quality of base calling obtained using various sequencing methods.

As a particular application of such, aspects of methods, systems, and media for predicting quality of base calling using various sequencing methods disclosed herein.

Other aspects of these aspects include corresponding computer systems, apparatus, and computer program product recorded on computer storage device(s), which, alone or in combination, configured to perform the actions of the methods. For a computer system configured or to be configured to perform operations or actions, the computer system has installed on it software, firmware, hardware, or their combinations that in operation cause the computer system to perform the operations or actions. For a computer program product configured or to be configured to perform operations or actions, the computer program product includes instructions that, when executed, by a hardware processor, cause the hardware processor to perform the operations or actions.

Further aspects, features, and advantages of the present disclosure, as well as the structure and operation of the various aspects of the present disclosure, are described in detail below with reference to the accompanying drawings.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the art(s) to make and use the aspects.

FIG. 1 illustrates a block diagram of a system for predicting quality of base calling, according to some aspects.

FIG. 2 is a flow chart illustrating a method for predicting quality of base-calling, according to some aspects.

FIG. 3A-3B is a block diagram of a look-up table of quality scores for predicting quality of base-calling, according to some aspects.

FIG. 4 illustrates a block diagram of a computer system for predicting quality of base calling, according to some aspects.

FIG. 5 illustrates tables of base calls and quality scores for generating the look-up table of quality scores for predicting quality of base-calling, according to some aspects.

FIGS. 6A-6B illustrate exemplary quality scores predictions across multiple channels (FIG. 6A) and within a single channel (FIG. 6B), according to some aspects.

FIGS. 7A-7D illustrates the prediction of quality scores across multiple channels (FIG. 7C) and within the same channel (FIG. 7D) in comparison with recalibrated quality scores, according to some aspects.

FIGS. 8A-8B illustrates accuracy of the prediction of quality scores across multiple channels and within the same channel, according to some aspects.

FIG. 9 is a schematic showing an exemplary linear single stranded library molecule, according to some aspects.

FIG. 10 is a schematic showing an exemplary linear single stranded library molecule, according some aspects.

FIG. 11 is a schematic of various exemplary configurations of multivalent molecules, according to some aspects.

FIG. 12 is a schematic of an exemplary multivalent molecule comprising a generic core attached to a plurality of nucleotide-arms, according to some aspects.

FIG. 13 is a schematic of an exemplary multivalent molecule comprising a dendrimer core attached to a plurality of nucleotide-arms, according to some aspects.

FIG. 14 shows a schematic of an exemplary multivalent molecule comprising a core attached to a plurality of nucleotide-arms, where the nucleotide arms comprise biotin, spacer, linker and a nucleotide unit, according to some aspects.

FIG. 15 is a schematic of an exemplary nucleotide-arm comprising a core attachment moiety, spacer, linker and nucleotide unit, according to some aspects.

FIG. 16 shows the chemical structure of an exemplary spacer (top), and the chemical structures of various exemplary linkers, including an 11-atom Linker, 16-atom Linker, 23-atom Linker and an N3 Linker (bottom), according to some aspects.

FIG. 17 shows the chemical structures of various exemplary linkers, including Linkers 1-9, according to some aspects.

FIG. 18 shows the chemical structures of various exemplary linkers joined/attached to nucleotide units, according to some aspects.

FIG. 19 shows the chemical structures of various exemplary linkers joined/attached to nucleotide units, according to some aspects.

FIG. 20 shows the chemical structures of various exemplary linkers joined/attached to nucleotide units, according to some aspects.

FIG. 21 shows the chemical structures of various exemplary linkers joined/attached to nucleotide units, according to some aspects.

FIG. 22 shows the chemical structure of an exemplary biotinylated nucleotide-arm, according to some aspects.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, method, and/or computer program product aspects, and/or combinations and sub-combinations thereof which enables predictive measurement of quality of base calling obtained using various sequencing methods disclosed herein. The techniques disclosed herein are useful for base-calling in next generation sequencing, and base-calling will be used as the primary example herein for describing the application of these techniques. However, such imaging analysis techniques may also be useful in other applications where spot-detection and/or charged coupled device (CCD) imaging is used.

In DNA sequencing, identifying the centers of polonies (which are often formed on beads) is sometimes referred to as primary analysis. Primary analysis involves the formation of a template for the flow cell. The template includes the estimated locations of all detected polonies in a common coordinate system. Templates are generated by identifying polony locations in all images in the first few flows of the sequencing process. The images may be aligned across all the images to provide the common coordinate system. Cluster or polony locations from different images may be merged based on proximity in the coordinate system. Once the template is generated, all further flow cell images are registered against it and the sequencing is performed based on the polony locations in the template.

After the actual polony centers are identified in each flow cell image, base calling may be performed based on the actual polony centers. Each polony of signals may be used to generate a single base call. The techniques disclosed herein may be used to predict the quality of base calling. A variety of algorithms exist for calculating the quality of base calling. These existing algorithms suffer from various shortcomings. For example, predicting quality score using neural networks may add computational burden to the processing hardware, and cause undesired delay in the estimation and downstream operations. As another example, quality score measurement that relies on downstream alignment information may not be able to provide predictive measurement of the base calls before the actual base calling has been made. Additionally, the accuracy and reliability of existing algorithms needs to be improved without sacrificing computational simplicity and processing time.

The techniques disclosed herein advantageously generate a look-up table based on two different quality scores, a standard quality score, and a conservative quality score, without the need of using complicated neural networks. The look-up table may be pre-determined, and the prediction of quality score may be conveniently and efficiently performed in parallel to the sequencing operations before any actual base calling has been made. The techniques disclosed herein also advantageously provide prediction of quality score across multiple channels and also within a single channel. Each channel may be provided a prediction of quality score independent of other channels, especially when the quality scores from different channels are different and may not be reliably reflected by the quality scores across multiple channels. Further, the techniques disclosed herein select predictors that are indicators of image quality of flow cells, and crowdedness of polonies so that they may be used to provide sensitive and accurate prediction of quality scores.

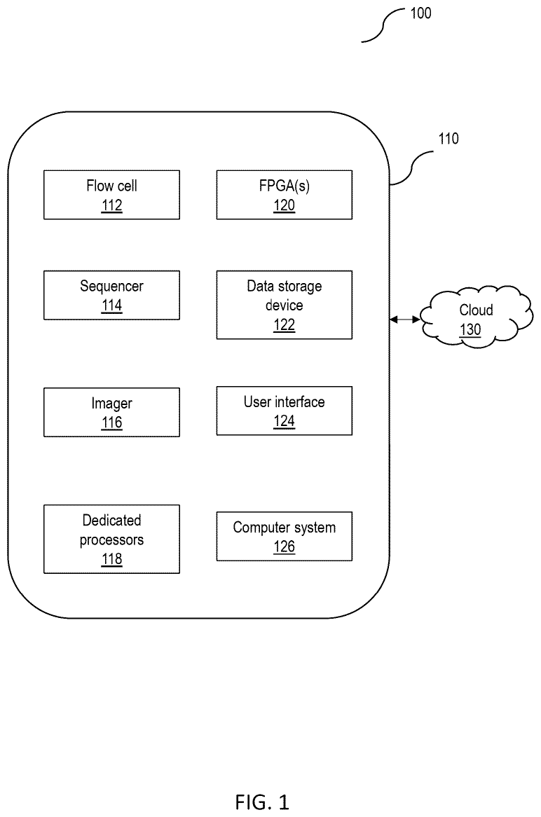

FIG. 1 illustrates a block diagram of a computer-implemented system 100, according to one or more aspects disclosed herein. The system 100 has a sequencing system 110 that includes a flow cell 112, a sequencer 114, an imager 116, data storage 122, and user interface 124. The sequencing system 110 may be connected to a cloud 130. The sequencing system 110 may include one or more of dedicated processors 118, Field-Programmable Gate Array(s) (FPGAs) 120, and a computer system 126.

In some aspects, the flow cell 112 comprises a solid support as disclosed herein. The support may have a coating thereon, e.g., a polymer coating disclosed herein. In some aspects, the flow cell 112 is configured to capture DNA fragments and form DNA sequences for base-calling on the flow cell. The sequencer 114 may be configured to flow a nucleotide mixture onto the flow cell 112, cleave blockers from the nucleotides in between flowing steps, and perform other steps for the formation of the DNA sequences on the flow cell 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths.

For example, each nucleotide base may be assigned a color. Different types of nucleotide may have different colors. Adenine may be red, cytosine may be blue, guanine may be green, and thymine may be yellow, for example. The color or wavelength of the fluorescent element for each nucleotide may be selected so that the nucleotides are distinguishable from one another based on the wavelengths of light emitted by the fluorescent elements.

The sequencer 114 may be configured to perform one or more sequencing methods disclosed herein, including but not limited to sequencing-by-avidite.

The imager 116 may be configured to capture images of the flow cell 112 after each flowing step. In an aspect, the imager 116 is a camera configured to capture digital images, such as an active-pixel sensor (CMOS) or a CCD camera. The camera may be configured to capture images at the wavelengths of the fluorescent elements bound to the nucleotides. The images may be called flow cell images. In some aspects, the imager 116 may include one or more optical systems disclosed herein.

In an aspect, the images of the flow cell may be captured in groups, where each image in the group is taken at a wavelength or in a spectrum that matches or includes only one of the fluorescent elements. In another aspect, the images may be captured as single images that captures all of the wavelengths of the fluorescent elements.

The resolution of the imager 116 controls the level of detail in the flow cell images, including pixel size. In existing systems, this resolution is very important, as it controls the accuracy with which a spot-finding algorithm identifies the polony centers. One way to increase the accuracy of spot finding is to improve the resolution of the imager 116 (e.g., by incorporating a higher-resolution camera), or improve the processing performed on images taken by imager 116. The methods described herein may detect polony centers in pixels other than those detected by a spot-finding algorithm. These methods allow for improved accuracy in detection of polony centers without increasing the resolution of the imager 116. The resolution of the imager may even be less than existing systems with comparable performance, which may reduce the cost of the sequencing system 110. In some aspects, the resolution of the imager may be the same as existing systems but achieve superior performance as compared to those existing systems due to the image processing.

The image quality of the flow cell images controls the base calling quality. One way to increase the accuracy of base calling is to improve the imager 116, or improve the processing performed on images taken by imager 116 to result in a better image quality. The methods described herein select image quality parameters and signal crowdedness as predictors of quality of base calling. Such predictors may be conveniently and efficiently calculated and made available when flow cell images are acquired. These methods allow for improved accuracy and reliability in predicting quality score of base calling without the trade-off of increased computational burden. Further, since the methods disclosed here are computationally less intensive than traditional methods so that the heat dissipation by the computer/processors may be easier to manage so that it is unlikely to cause undesired change to the proper chemistry of sequencing techniques disclosed herein. These methods may be advantageously performed in parallel in the computer-implemented system 100, without interference with or delay of existing sequencing workflow of the computer-implemented system 100. The results of predicted quality score may be available before actual base calling starts in the sequencing workflow.

The sequencing system 100 may be configured to predict quality score of base calling from one or more polonies based on the flow cell images. The operations or actions for predicting quality score of one or more polonies may be performed by the dedicated processors 118, the FPGA(s) 120, the computing system 126, or a combination thereof. One or more operations or actions in methods 200 300 disclosed herein may be performed by the dedicated processors 118, the FPGA(s) 120, the computing system 126, or a combination thereof. In some aspects, which operations or actions are to be performed by performed by the dedicated processors 118, the FPGA(s) 120, the computing system 126, or their combinations may be determined based on one or more of: a computation time for predicting quality score, a computation time for generating a look-up table, the complexity of computation in the specific operation(s), the need for data transmission between the hardware devices, or their combinations. Predicting quality score of base calling may be performed after the flow cell images are acquired but before actual base calling of the flow cell images is performed.

The computing system 126 may include one or more general purpose computers that provide interfaces to run a variety of program in an operating system, such as Windows™ or Linux™. Such an operating system typically provides great flexibility to a user.

In some aspects, the dedicated processors 118 may be configured to perform operations of predicting quality scores herein described herein. They may not be general-purpose processors, but instead custom processors with specific hardware or instructions for performing those steps. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general purpose computers. This may increase the speed at which the steps are performed and allow for real time processing.

In some aspects, the FPGA(s) 120 may be configured to perform operations in the methods for predicting quality scores herein. An FPGA is programmed as hardware that will only perform a specific task. A special programming language may be used to transform software steps into hardware componentry. Once an FPGA is programmed, the hardware directly processes digital data that is provided to it without running software. The FPGA instead uses logic gates and registers to process the digital data. Because there is no overhead required for an operating system, an FPGA generally processes data faster than a general-purpose computer. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 120 may be configured to perform the steps in parallel. For example, a number of FPGA(s) 120 may be configured to perform a processing step for an image, a set of images, or a polony location in one or more images. Each FPGA(s) 120 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over conventional systems may need to store the data before it may be processed, which may require more memory or accessing a computer system located in the cloud 130.

In some aspects, the data storage 122 is used to store information used in the predicting the quality scores. This information may include the images themselves or information derived from the images (e.g., pixel intensities, colors, etc.) captured by the imager 116. The DNA sequences determined from the base-calling may be stored in the data storage 122. As an example, the look-up table generated using method 300 may be stored in the data storage 122. Parameters identifying polony locations may also be stored in the data storage 122.

The user interface 124 may be used by a user to operate the sequencing system or access data stored in the data storage 122 or the computer system 126.

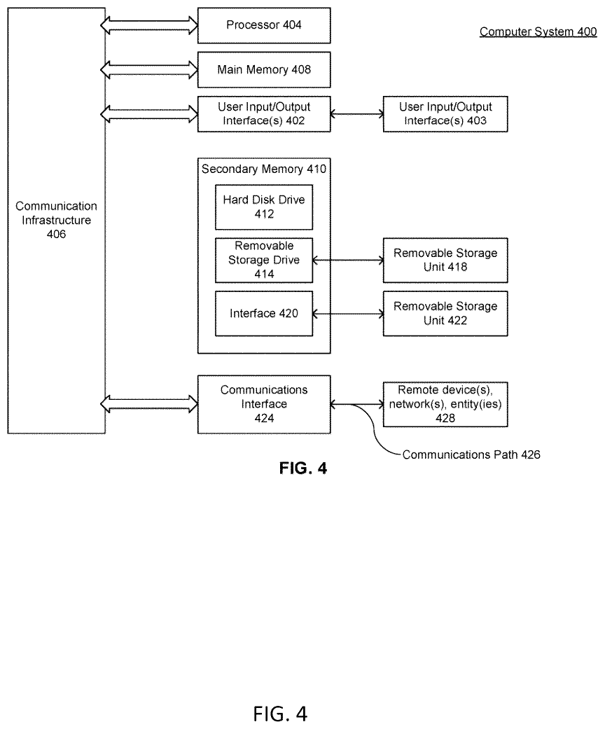

The computer system 126 may control the general operation of the sequencing system and may be coupled to the user interface 124. It may also perform steps in the prediction of quality scores and subsequence operations leading to actual base-calling. In some aspects, the computer system 126 is a computer system 400, as described in more detail in FIG. 4. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish necessary processing described above. In some aspects, when these elements are present together, the processing tasks are split between them. For example, the FPGA(s) 120 may be used to perform the methods for generating the look-up table, while the computer system 126 may perform other processing functions for the sequencing system 110. Those skilled in the art will understand that various combinations of these elements will allow various system aspects that balance efficiency and speed of processing with cost of processing elements.

The cloud 130 may be a network, remote storage, or some other remote computing system separate from the sequencing system 110. The connection to cloud 130 may allow access to data stored externally to the sequencing system 110 or allow for updating of software in the sequencing system 110.

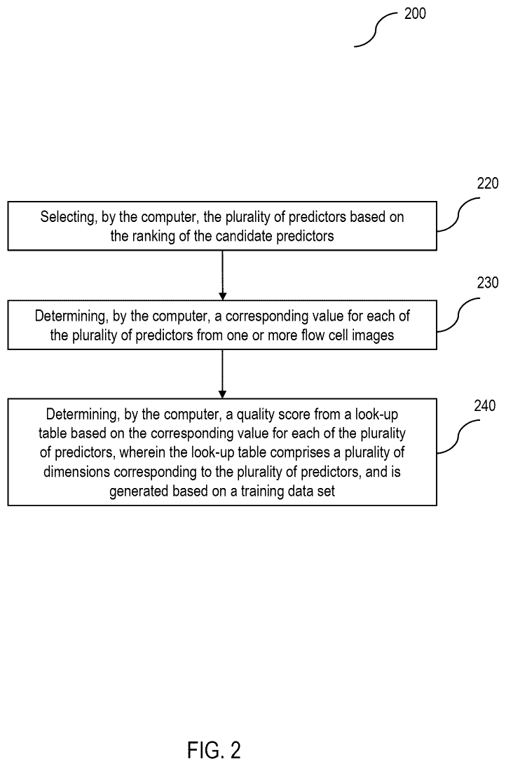

FIG. 2 shows a flow chart illustrating a method 200 for predicting quality of base calling in various sequencing applications such as DNA sequencing.

In some aspects, the method 200 comprises an operation of ranking candidate predictors based on their corresponding effects on quality of base calling. The candidate predictors may be indicative of the image quality of the one or more flow cell images. The candidate predictors may be indicative of polony density within the flow cell images or region(s) within the images. The candidate predictors may be indicative of signal errors that may negatively impact image quality for base calling, such errors may be based by color, phasing, pre-phasing, background noise, image distortion or transformation, etc. The candidate predictors may be indicative of characteristics that may influence image quality of the flow cell images including but not limited to the type of flow cells 112, the imager 116, the origin or of the DNA sequences, etc.

For example, the predictors are indicative of a signal to noise (SNR) ratio of one or more regions in flow cell image(s). As another example, the predictors are indicative of a contrast to noise (CNR) ratio of one or more regions in flow cell image(s). The corresponding effects of candidate predictors may be determined by their sensitiveness to CNR or accuracy of base calling of the flow cell images. With an identical CNR change in the image, the percentage of change in the first candidate predictor is greater than that of a second candidate predictor, the first candidate predictor may be ranked higher than the second candidate predictor. With an identical accuracy change in the base calling, if a percentage change in a first candidate predictor is greater than a second candidate predictor, the first candidate predictor may be ranked higher than the second candidate predictor. As another example, if the accuracy of base calling decreases as the density of polonies within a region of a fixed size increase, a candidate predictor may be selected that corresponds to the density of polonies. If sensitivity of two or more candidate predictors are substantially similar, with less than 1%, 2%, 3% or 5% difference, their ranking may be identical.

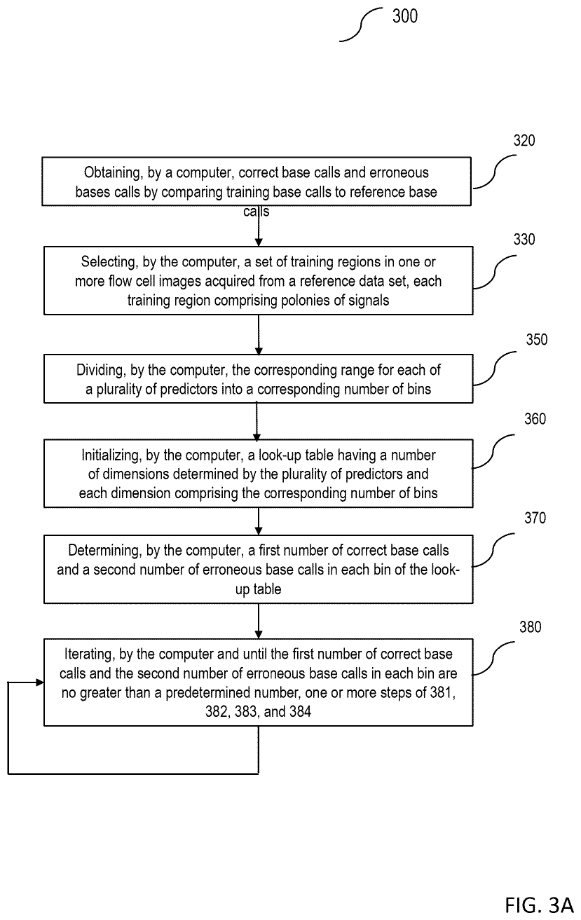

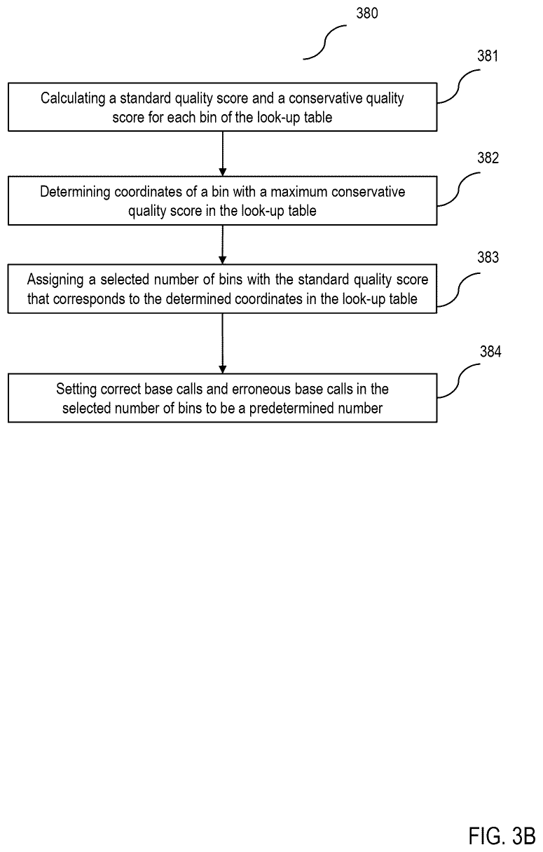

In some aspects, the method 200 comprises an operation 220 of selecting the predictors. The predictors may be selected based on the ranking of their corresponding effects on the quality of base calling. The quantity of selected predictors may include 2, 3, 4, 5, 6, or even more. Having more predictors may add additional computation time and complexity in generating the look-up table in the method 300 described in FIG. 3. The quantity of predictors may be manually selected based on a user indication received at the system 100. Alternative, the quantity of selected predictors may be automatically selected based on the characteristics of the system 100 or the specific custom application. For example, the quantity of predictors may be selected to be less than 7 to meet the time constraint in estimating the quality score or in generating the look-up table. Having more predictors may not necessarily improve the estimation of quality of base calling by a predetermined amount. The methods disclosed herein determines the number of predictors based on the trade-off between increasing computational burden and delay in generating the look-up table and quality scores and increased accuracy and sensitivity in quality score prediction.

In some aspects, for quality score prediction across multiple channels, the predictors disclosed herein include 3 different predictors. For example, the predictors include a first predictor of clarity, a second predictor of max_intensity, and a third predictor of low intensity median clarity. In some aspects, for quality score prediction across multiple channels, the predictors disclosed herein include 4 different predictors. For example, the predictors include a first predictor of clarity, a second predictor of max_intensity, a third predictor of low intensity median clarity, and a fourth predictor of phasing and prephasing.

In some aspects, the predictors disclosed herein include 3 or 4 predictors selected from a list including but is not limited to: a first predictor of clarity, a second predictor of max_intensity, a third predictor of low intensity median clarity, and a fourth predictor of phasing and prephasing.

The method 200 may further comprise operation 230 of determining a corresponding value for each predictor from one or more flow cell images. When the prediction of quality score is performed per cycle, the one or more flow cell images are within the same cycle, and the resulting predictors are determined per cycle.

The first predictor of clarity may include a ratio of a max_intensity to a second max_intensity. After determining a brightest channel among multiple channels, e.g., highest average intensity among 4 channels. The max_intensity may be obtained from the brightest channel. The max_intensity may be normalized. The normalization may be by a predetermined value, e.g., by 90th percentile brightest intensity within the channel, for flow cell images of each channel. The normalization may be performed during preprocessing operations of the flow cell images disclosed herein. Before obtaining the max_intensity, the flow cell image may be preprocessed using one or more of the preprocessing operations disclosed herein. The normalization may be performed after background subtraction, correction, and phasing and prephasing correction. After normalization, the signal intensity may be scaled to a pre-determined range, e.g., [0, 2000], [0, 2500], or [0, 3000]. The predetermined range may be determined to encode the range of normalized intensities in integers. To make the predictor changing inversely or negatively with the quality score, the scaled intensity may be negated and added to an intensity offset. The second predictor of max_intensity may be obtained as c−c1*(int/int_norm), wherein c and c1 may be identical or different integers, int is the intensity of the polony, and int_norm is the intensity used for normalization of intensity int. For example, int_norm may be the brightest intensity of the flow cell image after background subtraction and color correction. The max_intensity may be obtained for each polony within the flow cell image in a cycle. In other words, the max_intensity may be per polony-cycle. The second max_intensity may be determined similarly as the max_intensity disclosed herein, but with respect to the second brightest channel among multiple channels, e.g., the second highest average intensity among 4 channels. The second max_intensity may be obtained for each polony within the flow cell image in a cycle. In other words, the second max_intensity may be per polony-cycle. As such, the first predictor of clarity may be per polony-cycle. The second predictor of max_intensity may also be per polony-cycle.

For quality score prediction across multiple channels, the calculation of max_intensity for the second predictor of max_intensity is the same as disclosed herein in the calculation of the first predictor of clarity.

In some aspects, the value of predictors and the accuracy of base calling satisfies a similar pattern, which is the smaller the value of a predictor, the more accurate the base calling. In other words, the value of the predictor is negatively or inversely correlated with the accuracy of base calling. For example, the first predictor of clarity comprises an inverse of the ratio of the max_intensity to the second max_intensity. As another example, the second predictor of max_intensity includes a fixed offset value, e.g., about 2000, that is added to the negative max_intensity. The fixed offset value may be customized to provide optimal quality score prediction results for different sequencing systems 110 in which one or more subunits of the system 110 is different from a reference sequencing system 110, e.g., a next generation sequencing system-by-avidite as disclosed herein.

The third predictor of low intensity median clarity is indicative of density of polonies in the flow cell images. The third predictor of low intensity median clarity comprises a median clarity for a selected number of polonies in a region that may be called a subtitle. A flow cell image may be separated into multiple tiles, and each tile is an imaging area that may have multiple subtiles, and each subtile may have an equal or different number of polonies. As an example, a flow cell may include 424 tiles, and depending on the density of polonies in the tile, a total number of polonies may be 3 to 5 million of polonies per tile. A flow cell image disclosed herein may be an image of a single tile. Alternatively, a flow cell image disclosed herein may be an image of multiple tiles. To make the third predictor compatible with the inverse or negative pattern described above, it is inversely proportional to the median clarity of selected polonies per tile-cycle in the one or more flow cell images. The selected number of polonies are dim polonies selected from the flow cell image. A dim polony may have an intensity lower than a pre-determined percentage of the brightest signal intensity in a tile or a subtile. For example, the pre-determined percentage may be 15%, 20%, 25%, 30%, or any other percentages lower than 45%. Alternatively, a dim polony belongs to the darkest population of polonies with a pre-selected percentage. The pre-selected percentage may be 8%, 10%, 12%, or 15% of the total population of polonies within the subtile or tile. A median of all clarity values from the dim tiles generates the median clarity. The third predictor of low intensity median clarity is further inversed to obtain the predictor that is smaller when the quality score estimation is more accurate. The third predictor of low intensity median clarity may be calculated as c*median (max_r2/max_r), wherein c is a constant, max_r is maximal signal intensity of the dim polonies within a selected region of the flow cell image, and max_r2 is the second maximal signal intensity of the dim polonies within the same selected region. The third predictor of low intensity median clarity may be calculated as c/median (max_r/max_r2), wherein c is a constant, max_r is maximal signal intensity of the dim polonies within a selected region of the flow cell image, and max_r2 is the second maximal signal intensity of the dim polonies within the same selected region.

The fourth predictor of phasing and prephasing may be determined as an average percentage of phasing and prephasing in a selected number of polonies in a region of the flow cell images. The region of the flow cells are tiles as described herein. The corresponding value of the fourth predictor of phasing and prephasing is based on multiple polonies in the flow cell image. In other words, the fourth predictor of phasing and prephasing is per tile-cycle.

The method 200 may further comprise one or more preprocessing operations for preprocessing the flow cell images, before or after operation of ranking the predictors. Such preprocessing operations includes one or more of: (1) background subtraction; (2) color correction; (3) phasing or prephasing correction; and (4) and normalization. The preprocessing operations are configured to improve image quality and reduce errors that might be cause by noise and biases introduced during imaging using the sequencer and/or imager 116.

The flow cell images disclosed herein may be after one or more of the preprocessing steps disclosed herein. The flow cell images may comprise images acquired from multiple channels. For example, the flow cell image may include images from all 4 different channels. In some aspects, operation (4) normalization may include normalization of intensities of flow cell images, e.g., in each channel by 90^{th} percentile of the corresponding channel.

One channel disclosed herein corresponds to a wavelength spectrum of a fluorescent element representing a nucleotide base in a set of nucleotide bases.

The method 200 may further comprises operation 240, which is determining a quality score from a look-up table based on the corresponding value for each of the plurality of predictors. The look-up table may be generated using method 300 in FIG. 3. The look-up table may include a plurality of dimensions, each dimension corresponding to a selected predictor. The look-up table may be prepopulated with values by using a reference base call set and a training data set.

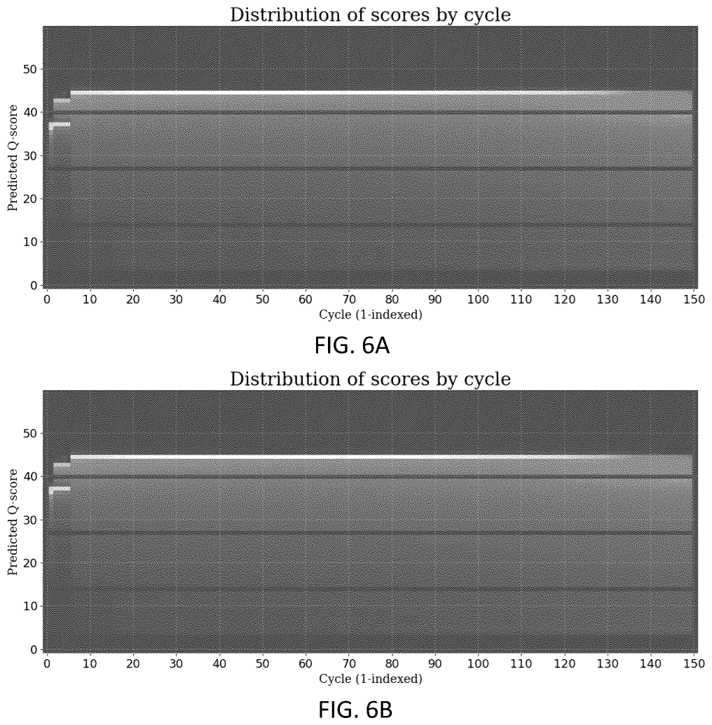

FIGS. 6A-6B show predicted quality scores generated using the methods, systems, and media disclosed here. The predicted quality scores are plotted for each cycle from cycle 1 to cycle 150. FIG. 6A shows predicted quality score across four different channels, and FIG. 6B shows predicted quality score for each individual channel. The spread of light colors above the dark/black background shows that the predicted quality score spread in a range from about 35 to 45 or more in earlier cycles, e.g., cycle 6 to about cycle 120, and the average quality score slightly decreases in later cycles, e.g., cycles 130-150.

In some aspects, unlike traditional quality score that predict a same quality score for all channels, the method 200 may be used for predicting quality of base calling for each individual channels. Such aspects advantageously remove the need to normalize intensity of flow cell images but instead may work with raw image intensity of a specific channel, so as to provide computationally simplicity and channel-specific estimation over traditional methods. Further, such aspects may work with a larger range of image intensity in the flow cell images for determine base calling, thereby providing a larger dynamic range and more accurate quality estimation than traditional methods.

In such aspects, the preprocessing operations of predicting the quality score within a single channel is different from that of prediction of quality score across multiple channels. In some aspects, preprocessing operations of the flow cell images may comprise one or more of: (1) background subtraction; (2) color correction; and (3) phasing or prephasing correction, but not (4) normalization. The preprocessing operations may also include image registration of a selected cycle to the template as disclosed herein. In some aspects, one or more of the preprocessing operations is performed with respect to flow cell images from the same channel. In some aspects, one or more of the preprocessing operations is performed with respect to flow cell images from different channels. In some aspects, (3) phasing and prephasing correction may be performed with respect to individual channels so that each flow cell image per channel may have its independent phasing and/or prephasing correction. In other aspects, a median or average of the phasing and/or prephasing correction across different channels may be applied to all the flow cell images from different channels. In some aspects, the preprocessing operations may include extracting polonies from normalized flow cell images and register them within a common coordinate system. After normalization, the image intensity of different channels may be in similar intensity ranges. In other words, normalization reduces the variation in the ranges of image intensities across channels. An example technique for extracting the plurality of polonies in a flow cell is described in U.S. Pat. No. 11,200,446, which is hereby incorporated by reference in its entirety. The image intensities may be determined as described in, for example, U.S. Pat. No. 11,200,446, incorporated by reference herein in its entirety.

Operations 220, 230, and 240 and other operations disclosed herein in relation to method 200 are similar for predicting quality score across multiple channels or within a single channel. However, the second predictor of max_intensity is used in the methods 200 for predicting the quality across multiple channels, and it is replaced by the second predictor of max_cc_intensity when the prediction is for a single channel. The replacement of max_intensity to max_cc_intensity may be only within calculation of the second predictor, the calculation of predictors other than the second predictor may stay the same as those disclosed herein for making predictions across multiple channels.

Alternatively, in some aspects, the replacement of max_intensity to max_cc_intensity may be in two or more predictors, for example, in the first predictor of clarity and the second predictor. The other predictors without using max_intensity may stay the same as the as those disclosed herein for making predictions across multiple channels.

In some aspects, for quality score prediction within a single channel, the predictors disclosed herein include 3 different predictors. For example, the predictors include a first predictor of clarity, a second predictor of max_cc_intensity, and a third predictor of low intensity median clarity. In some aspects, for quality score prediction within a single channel, the predictors disclosed herein include 4 different predictors. For example, the predictors include a first predictor of clarity, a second predictor of max_cc_intensity, a third predictor of low intensity median clarity, and a fourth predictor of phasing and prephasing. For quality score estimation within a single channel, the second predictor of max_cc_intensity is calculated differently from that of the second predictor for multiple channels. In some aspects, the predictors disclosed herein include 3 or 4 predictors selected from a list including but is not limited to: a first predictor of clarity, a second predictor of max_cc_intensity, a third predictor of low intensity median clarity, and a fourth predictor of phasing and prephasing.

In some aspects, the max_cc_intensity may be calculated as c′−c1′*(int_raw), wherein c′ and c1′ are identical or different integers, int_raw is the raw intensity of the polony without normalization. The raw intensity may be after background subtraction, color correction, phasing and prephasing correction, or their combinations. The signal intensity may be scaled to a pre-determined range, e.g., [0, 300], [0, 400], or [0, 500]. The predetermined range may be determined to encode the range of raw intensities in integers. To make the predictor changing inversely or negatively with the quality score, the scaled intensity may be negated and added to an intensity offset.

For quality score within a single channel, when max_intensity is replaced by max_cc_intensity in the first predictor of clarity, the first predictor of clarity may include a ratio of a max_cc_intensity to a second max_cc_intensity. After determining a brightest channel among multiple channels, e.g., highest average intensity among 4 channels. The max_cc_intensity may be obtained from the brightest channel. The max_cc_intensity may be without normalized by a predetermined value, e.g., by 90^{th} percentile brightest intensity within the channel, for flow cell images of each channel. Before obtaining the max_cc_intensity, the flow cell image may be preprocessed using one or more of the preprocessing operations disclosed herein, e.g., background subtraction, correction, and phasing and prephasing correction. After preprocessing, the signal intensity may be scaled to a pre-determined range, e.g., [0, 300], [0, 400], or [0, 500]. The predetermined range may be determined to encode the range of intensities in integers. To make the first predictor changing inversely or negatively with the quality score, the scaled intensity may be negated and added to an intensity offset. The first predictor of max_cc_intensity may be obtained as c−c*(int), wherein c is an integer, int is the intensity of the polony. The max_cc_intensity may be obtained for each polony within the flow cell image in a cycle. In other words, the max_cc_intensity may be per polony-cycle. The second max_cc_intensity may be determined similarly as the max_cc_intensity disclosed herein, but with respect to the second brightest channel among multiple channels, e.g., the second highest average intensity among 4 channels. The second max_cc_intensity may be obtained for each polony within the flow cell image in a cycle. In other words, the second max_cc_intensity may be per polony-cycle. As such, the first predictor of clarity may be per polony-cycle. The second predictor of max_cc_intensity may also be per polony-cycle.

For quality score within a single channel, when max_intensity is replaced by max_cc_intensity in the second predictor of max_intensity. The calculation of max_cc_intensity may be the same as disclosed in the calculation of the first predictor of clarity.

In some aspects, the value of predictors and the accuracy of base calling satisfies a similar pattern, which is the smaller the value of a predictor, the more accurate the base calling. In other words, the value of the predictor is negatively or inversely correlated with the accuracy of base calling. For example, the first predictor of clarity comprises an inverse of the ratio of the max_cc_intensity to the second max_cc_intensity. As another example, the second predictor of max_cc_intensity includes a fixed offset value, e.g., about 2000, that is added to the negative max_cc_intensity. The fixed offset value may be customized to provide optimal quality score prediction results for different sequencing systems 110 in which one or more subunits of the system 110 is different from a reference sequencing system 110, e.g., a next generation sequencing system-by-avidite as disclosed herein.

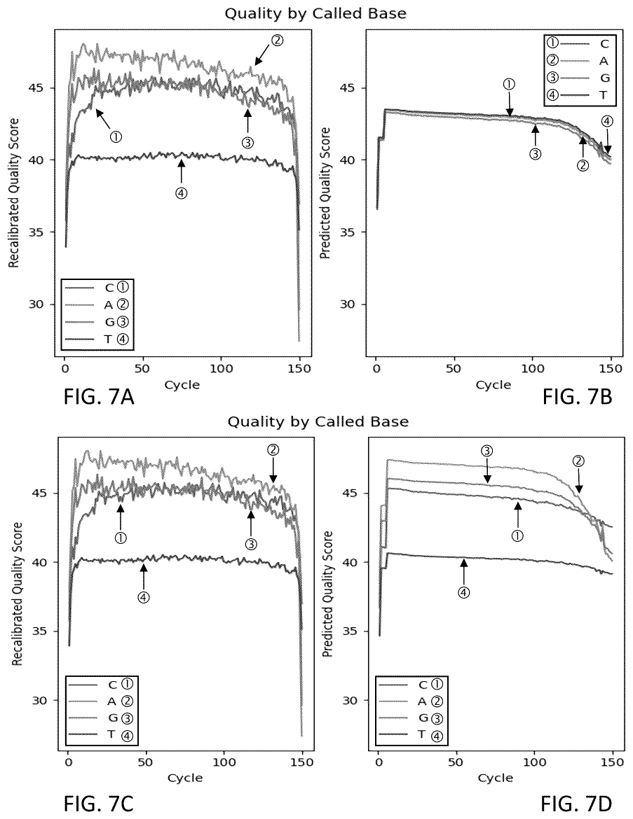

FIGS. 7A-7D show the predicted quality score generated using the methods disclosed herein in comparison with recalibrated quality scores. FIGS. 7A-7B show the average recalibrated quality score and the average predicted quality score, respectively. The predicted quality scores are generated using prediction across all 4 channels. FIG. 7B illustrates that the average predicted quality score is substantially identical across different channels. FIG. 7A illustrate that the recalibrated average quality score is different across channels, and the T channel has lowest quality score among the four channels. The average quality score of channel A has relatively higher quality score than the other three channels. FIGS. 7C-7D show the average recalibrated quality score and the average predicted quality score, respectively, obtained using the same flow cell images. The predicted quality scores are generated using prediction within a single channel so that the quality score prediction may be channel specific. FIG. 7D illustrates that the average predicted quality scores at different cycles are different in 4 channels. FIG. 7C illustrate that the recalibrated average quality scores are different across channels, and the T channel has lowest quality score among the four channels. Unlike FIG. 7B, such difference across channels has been reflected in the predicted average quality score in FIG. 7D.

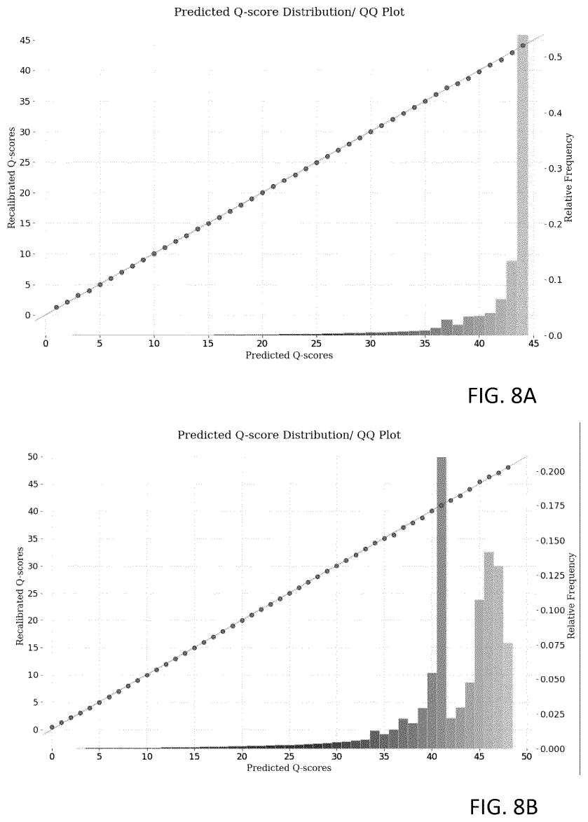

FIGS. 8A-8B show the accuracy of predicted quality score obtained using the technologies disclosed herein. The quality score recalibration is a standard technique for evaluating predicted quality score. Various recalibration methods may be used. For example, for a specific quality score, all the errors and correct base call counts for that specific quality score, and get recalibrated quality score, which −10*log 10 (error rate). After a mapping of the quality score to the recalibrated score, accuracy may be calculated. In this aspect, FIG. 8A show the comparison between predicted quality scores with the recalibrated quality scores. The predicted quality score is across all 4 channels. The predicted quality score is substantially aligned with the recalibrated score in all quality score values and in different cycles. The histogram of predicted quality score value shows a peak between Q40 and Q45. FIG. 8B show the comparison between predicted quality scores with the recalibrated quality scores within a single channel. The predicted quality score is substantially aligned with the recalibrated score in all quality score values and in different cycles. The histogram of predicted quality score value shows a peak between Q40 and Q45.

FIG. 3 shows a flow chart illustrating a method 300 for generating a look-up table for predicting quality of base calling in DNA sequencing. The look-up table may have multiple dimensions and each dimension corresponding to a selected predictor. The method 300 may be performed with a selected number of cycles of sequencing, so that a lock-up table may be generated that may be used for different cycles. The selected number may be 100, 150, 200, or any integer number. The selected number may be 1 so that the look-up table is generated from a specific cycle of sequencing.

The method 300 may comprise an operation of obtaining correct base calls and erroneous bases calls by comparing training base calls to a reference base call set. The training base calls are generated using a reference data set with a reference base call set. The reference data set may be a pre-selected genome or DNA sequence. As a non-limiting example, the reference set may include HG001, HG005, HG38 consensus human genome, or there combinations.

Before the operation of obtaining correct base calls and erroneous bases calls, the method may comprise operation 220, the operation of ranking the predictors as disclosed in relation to FIG. 2 as disclosed herein.

Before the operation of obtaining correct base calls and erroneous bases calls, the method may further comprise filtering the reference base call set by removing base calls from positions in the genome(s) of the reference data set with pre-determined variants. The method may further comprise filtering the reference base call set by removing base calls from positions in the genome(s) of the reference data set with pre-determined alignment errors. The filtering of the reference data set may advantageously remove error sources that are not intrinsic to the system 100, so that errors in base calling may be more accurately and reliably attributed to the system 100 and the subunits contained therein.

Before the operation of obtaining correct base calls and erroneous bases calls and after the filtering operation disclosed herein, the method 300 may further comprise obtaining training base calls in the flow cell images of the reference data set. The flow cell images may be acquired using the system 100. And the training base calls are made by using the flow cell images.

The method 300 may further comprise an operation 320 in which, a set of training regions in each flow cell image is selected, and each training region may contain multiple polonies of signals. The training regions may be obtained from an identical cycle or different cycles of sequencing. The training regions may be from the same run or different runs. The training regions may be from Read 1, Read 2, or both. As an example, each flow cell image may include a number of tiles, each tile representing an imaging area on the flow cell. Each tile may be further divided into a grid of subtiles. For example, a flow cell image may have 424 tiles, a flow cell image may be generated for each tile during a read in a cycle for a channel. The training region may include subtiles that in sum equivalent to 48 tiles of polonies. Each tile may include about 1 to 10 million of polonies. And the flow cell images may be obtained from 150 cycles, 2 reads, and 20 to 30 runs in all 4 channels. The amount of training polonies in training regions may be on the scale 10\hat{ }10 or more. For each training polony, its predictors may be determined, and its quality scores may be calculated, the base calling may be made and aggregated in the correct bin of the look-up table. There may be a computational load that requires a computer system 126 disclosed herein, one or more dedicated processors 118, and/or FPGA(s) 120. Each region disclosed herein may be a subtile, and each subtile may include a number of polonies in the range of 5,000 to 200,000. As a non-limiting example, each subtile may include a number of polonies in the range of 10,000 to 80,000. As another non-limiting example, each subtile may include a number of polonies in the range of 20,000 to 60,000.

To reduce computation time of performing the methods disclosed herein in a general purpose computer, some or all of the operations may be performed by the dedicated processors 118, and/or FPGA(s) 120. Performing some or all of the operations in the dedicated processors 118, and/or FPGA(s) may advantageously help with the heat dissipated by the general purpose computers which may adversely affect the temperature of the flow cells, thereby causing undesired problems in the chemistry of sequencing disclosed herein.

For quality scores across multiple channels or quality score of a single channel, the training regions include regions from multiple channels, for example, all 4 channels, 3 channels, or 2 channels.

The method 300 further comprises operation 330, which is determining a corresponding range for each predictor using the training polonies in the set of training regions. The corresponding range may encompass possible values of the predictor in all training polonies of the training regions, either from a single cycle, or from multiple different cycles. The corresponding range may start from a minimal value of the predictor in all the training polonies and end with the maximal value of the predictors.

For quality scores across multiple channels, the flow cell images may have been preprocessed using the operations disclosed herein, so that the range may be post-processed range of the training polonies. For quality score within a single channel, the flow cell image from the single channel may include raw image intensity, so that the range may be for the raw intensities of the training regions.

After the corresponding range is determined in operation 330, the method 300 may further include an operation of dividing the corresponding range for each predictor into a corresponding number of bins. The corresponding number of bins do not need to be identical for two or more predictors. The increased number of bins will result in more computational complexity and time consumption in generating the look-up table. At the same time, the increased bins provides better resolution within the range, thus it may provide more accuracy to the look-up table. As a non-limiting example, the number of bins may be 50 for different predictors. With 4 different predictors, the look-up table will be 50×50×50×50 in size. In some aspects, the number of bins may be 100. In some aspects, the number of bins may be an integer number in the range of 20 to 150. In some aspects, the number of bins may be an integer number in the range of 40 to 100. In some aspects, the number of bins may be an integer number in the range of 40 to 60.

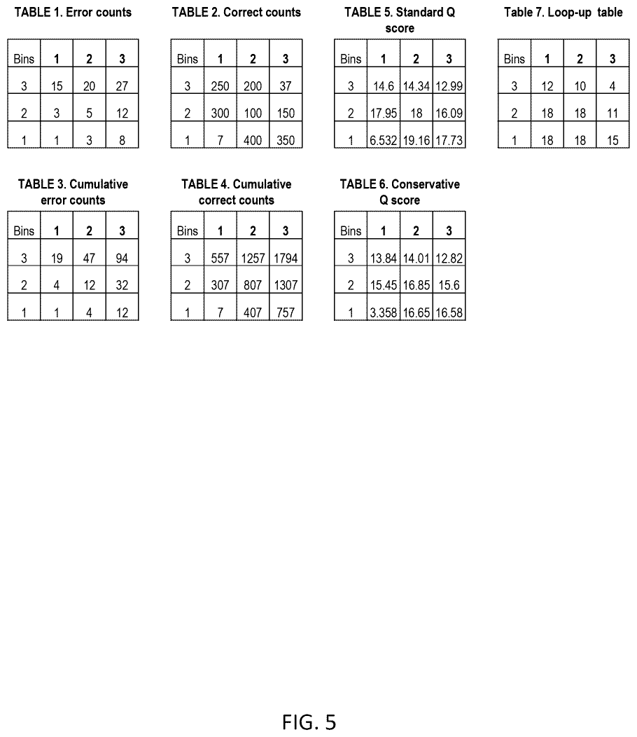

The method 300 may further comprise operation 350, which is determining a first number of correct base calls and a second number of erroneous base calls in each of bin for each of the plurality of predictors. For each polony in the training regions (e.g., for all the cycles or in a cycle), the corresponding values of the predictors for this polony may be obtained, the values may be used to find the corresponding bin. The base call of this polony, either correct or erroneous, by comparing to the reference base call, may be added to the correct or erroneous count of this bin. And the process may be repeated for all the polonies to generate the correct and erroneous counts for each bin. The correct base call counts may be saved in a correct base call table which is the same number of bins in each dimension as the look-up table. Similarly, the erroneous base call counts may be saved in an erroneous base call table, which is also the same size as the look-up table. Exemplary correct and erroneous base call tables are shown in TABLE 2 and TABLE 1, respectively in FIG. 5.

Using the correct base calls and erroneous based calls determined in operation 440. The method 300 may further comprise determining a first cumulative number of correct base calls and a second cumulative number of erroneous base calls in each of bin for each of the plurality of predictors. The cumulative number of correct base calls including a sum of correct base calls from all the bins that has a coordinate smaller than or equal to the coordinate of the current bin. The cumulative number of erroneous base calls may be determined in a similar fashion. For example, if a bin has a coordinate of (i*, j*), then the base calls from all the bins whose coordinate ranges from 1 to i* and from 1 to j* are summed up to generate the cumulative number of correct or erroneous base calls for bin (i*, j*). The cumulative number of correct base calls and erroneous calls may be saved in a separate table, each table having an identical size as the look-up table. Exemplary cumulative correct and erroneous base call tables are shown in TABLE 4 and TABLE 3, respectively in FIG. 5. In this example, each table is with 2 dimensions, and 3 bins in each dimension.

The method 300 may further comprise operation 380, which is iterating, by the computer and until correct base calls and erroneous base calls in each bin are no greater than a predetermined number, one or more of the following steps: step 381 calculating a standard quality score and a conservative quality score for each bin of the look-up table; step 382 finding coordinates of a bin with a maximum conservative quality score in the look-up table; step 383 assigning a selected number of bins with the standard quality score that corresponds to the coordinates in a look-up table; and step 384 setting correct base calls and erroneous base calls in the selected number of bins to be a predetermined number. In some aspects, the pre-determined number may be 0. In other aspects, the predetermined number may be a small number, e.g., 2, 3, 4, or 5, that is not greater than 10.

Assuming there are 4 selected predictors, and each predictor has an identical number of n bins. In step 381, for all the bins in the look-up table, the standard quality score may be calculated using the cumulative correct and erroneous base call counts using equations (1) to (6). The cumulative erroneous base call count may be calculated as:

$$err ( i , j , k , m ) = {\sum }_{{i}^{′} , {j}^{′} , {k}^{′} , {m}^{′}} {err}_{count} ( {i}^{′} , {j}^{′} , {k}^{′} , {m}^{′} ) \qquad ( 1 )$$

where err ( ) is the cumulative erroneous base call counts, and err_{count}( ) is the erroneous count in individual bins, and i′ is in the range of [0, i], j′ is in the range of [0, j], k′ is in the range of [0, k], and m′ is in the range of [0, m], wherein i, j, k, and m may be in the range of [0, n].

Similarly, the cumulative correct base call count may be calculated as:

$$corr ( i , j , k , m ) = {\sum }_{{i}^{′} , {j}^{′} , {k}^{′} , {m}^{′}} {corr}_{count} ( {i}^{′} , {j}^{′} , {k}^{′} , {m}^{′} ) \qquad ( 2 )$$

The standard error rate, e(i,j,k,m) may be obtained as:

$$e ( i , j , k , m ) = \frac{cs + err ( i , j , k , m )}{cs + err ( i , j , k , m ) + corr ( i , j , k , m )} \qquad ( 3 )$$

The conservative error rate, ec(i,j,k,m) may be obtained as

$$ec ( i , j , k , m ) = \frac{cc + err ( i , j , k , m )}{cc + err ( i , j , k , m ) + corr ( i , j , k , m )} \qquad ( 4 )$$

Where cs is correction number that may be a positive constant, and cc is a different correction number that may be a different positive constant that is greater than cs. As a non-limiting example, cs may be 1, and cc may be 5. Correspondingly, the standard quality score Q(i,j,k,m) and the conservative quality score Qc(i,j,k,m) may be obtained from the standard error rate, or the conservative error rate using equations (5) or (6) as below:

$$Q ( i , j , k , m ) = - 10 * log 10 ( e ( i , j , k , m ) \qquad ( 5 )$$

$$Qc ( i , j , k , m ) = - 10 * log 10 ( ec ( i , j , k , m ) \qquad ( 6 )$$

Exemplary conservative and standard quality scores are shown in TABLE 6 and TABLE 5 in FIG. 5.

In step 382, the coordinates (i*, j*,k*, m*) of the bin with highest conservative quality score may be determined. In step 383, all the bins with coordinates i, j, k, and m less than or equal to i*, j*, k*, and m*, respectively, may be assigned with the standard quality score value of bin (i*, j*,k*, m*). Then in step 384, the error base calls and correct base calls in all the bins with assigned quality values are set to a predetermined value. Exemplary error and correct base calls are in TABLE 1 and TABLE 2 in FIG. 5. A non-limiting example of this predetermined value is 0. The updated error base calls and correct base calls may be used to update the cumulative erroneous and correct base call counts using equations (1) and (2) for these bins. Subsequently, the corresponding standard and conservative error rates may be updated using equations (3) and (4). The standard and conservative quality scores may then be updated using equations (5) and (6).

After the updates, step 381 may be performed thereby starting a new iteration of steps 381-384. When all the error counts and correct counts of base calls in the bins are no greater than the predetermined threshold, the iteration of operation 380 stops, and the look-up table may be generated with a quality score in each of its bins. An exemplary look-up table with 2 dimensions, and 3 bins in each dimension is shown in TABLE 7 of FIG. 5. This look-up table is simplified, and the quality score may not correspond to actual quality scores predicted with the system 100.

### Example Generation of the Look-Up Table for Prediction of Quality Score of a Single Channel

In an exemplary aspect, the method 300 disclosed herein is used to generate a look-up table for a HG001 human genome. Known positions for common variants are filtered out from the human genome, and regions that present uncommon patterns like CGCGCG, are also removed. Flow cell images are acquired using the system 100. The flow cell images come from 20 runs, 2 reads, and 150 cycles of 4 different channels. In each cycle per channel per read, flow cell images acquired equal the total number of tiles on the flow cell. Among these images, a region (subtile) in some or all of the tiles are selected from each flow cell image. Each subtile may comprise 30 k polonies. Three predictors, the max_intesity, clarity, and low intensity median clarity are selected. The values of the first two predictors are calculated per colony-cycle. The values of the last predictor are calculated per tile-cycle. The values of the predictors are calculated after preprocessing except that the max_intensity. The preprocessing includes: background subtraction, color correction, phasing and prephasing correction, and normalization using 90^{th} percentile of the brightest intensity of the flow cell image in the corresponding channel. The max_intensity is the raw image intensity without normalization. And the range of the values for each predictor is estimated based on the different values in the selected training regions of all the cycles. The range for second predictor of max_intensity and other predictors disclosed herein may encompass all the possible values in the training regions of different cycles. In some aspects, since the range is estimated, it may not encompass all the possible values either in the training regions. After the look-up table is generated, when a predictor has a value that is outside the range of the look-up table, the method 200 may select a bin that is closest to the value for locating its quality score. Then, each range is evenly separated into 50 bins. So, the look-up table has 50×50×50 in size. Base calling is performed using the system 100, and the base calls coming from polonies whose predictor values fell into a bin, are counted into either the erroneous or correct base call counts of that bin. For example, bin (25, 25, 25) may have 10% erroneous base calls among the total number base calls. The look-up table has an average quality score of Q40.

### Example Prediction of Quality Score

In an exemplary aspect of method 200, flow cell images are acquired using the system 100. The flow cell images come from 30 runs, 2 reads, and 150 cycles with only signal intensities from a single channel. The raw image intensities of the flow cell images without preprocessing are used. In other words, the raw image intensities of the flow cell images without phasing and prephasing correction and normalization are used for calculating the value for each of three predictors, i.e., the max_intesity, clarity, and low intensity median clarity. The value is per polony-cycle for the max_intensity and clarity. The value is per-tile-cycle for low intensity median clarity. After the value determined, for each polony, three different values of the predictors are used as coordinate to find a bin in the look-up table, and the quality score for the polony is the quality score at the coordinate of the look-up table. A coordinate of [1000, 0.5, 200] leads to bin [25, 25, 25] of the look-up table, and the quality score for base calling using the polony is Q40.

### Example Computer System

Various aspects of the method 200 and 300 may be implemented, for example, using one or more computer systems, such as computer system 400 shown in FIG. 4. One or more computer systems 400 may be used, for example, to implement any of the aspects discussed herein, as well as combinations and sub-combinations thereof.

Computer system 400 may include one or more hardware processors 404. The hardware processor 404 may be central processing unit (CPU), graphic processing units (GPU), or their combination. Processor 404 may be connected to a bus or communication infrastructure 406.

Computer system 400 may also include user input/output device(s) 403, such as monitors, keyboards, pointing devices, etc., which may communicate with communication infrastructure 406 through user input/output interface(s) 402. The user input/output devices 403 may be coupled to the user interface 124 in FIG. 1.

One or more of processors 404 may be a graphics processing unit (GPU). In an aspect, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, and/or producing results of other proof-of-work computations for some blockchain-based applications, for example. With capabilities of general-purpose computing on graphics processing units (GPGPU), the GPU may be particularly useful in at least the image recognition and machine learning aspects described herein.

Additionally, one or more of processors 404 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors and/or other logic to facilitate such acceleration.

Computer system 400 may also include a data storage device such as a main or primary memory 408, e.g., random access memory (RAM). Main memory 408 may include one or more levels of cache. Main memory 408 may have stored therein control logic (i.e., computer software) and/or data.

Computer system 400 may also include one or more secondary data storage devices or secondary memory 410. Secondary memory 410 may include, for example, a main storage drive 412 and/or a removable storage device or drive 414. Main storage drive 412 may be a hard disk drive or solid-state drive, for example. Removable storage drive 414 may be a floppy disk drive, a magnetic tape drive, a compact disk drive, an optical storage device, tape backup device, and/or any other storage device/drive.

Removable storage drive 414 may interact with a removable storage unit 418.

Removable storage unit 418 may include a computer usable or readable storage device having stored thereon computer software and/or data. The software may include control logic. The software may include instructions executable by the hardware processor(s) 404. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities or other approaches for allowing computer programs and/or other instructions and/or data to be accessed by computer system 400. Such means, devices, components, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communication path 426, which may be wired and/or wireless (or a combination thereof), and which may include any combination of LANs, WANs, the Internet, etc. Control logic and/or data may be transmitted to and from computer system 400 via communication path 426. In some aspects, communication path 426 is the connection to the cloud 130, as depicted in FIG. 1. The external devices, etc. referred to by reference number 428 may be devices, networks, entities, etc. in the cloud 130.

Computer system 400 may also be any of a personal digital assistant (PDA), desktop workstation, laptop or notebook computer, netbook, tablet, smart phone, smart watch or other wearable, appliance, part of the Internet of Things (IoT), and/or embedded system, to name a few non-limiting examples, or any combination thereof.

It should be appreciated that the framework described herein may be implemented as a method, process, apparatus, system, or article of manufacture such as a non-transitory computer-readable medium or device. For illustration purposes, the present framework may be described in the context of distributed ledgers being publicly available, or at least available to untrusted third parties. One example as a modern use case is with blockchain-based systems. It should be appreciated, however, that the present framework may also be applied in other settings where sensitive or confidential information may need to pass by or through hands of untrusted third parties, and that this technology is in no way limited to distributed ledgers or blockchain uses.

Computer system 400 may be a client or server, accessing or hosting any applications and/or data through any delivery paradigm, including but not limited to remote or distributed cloud computing solutions; local or on-premises software (e.g., “on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); and/or a hybrid model including any combination of the foregoing examples or other services or delivery paradigms.

Any applicable data structures, file formats, and schemas may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

Any pertinent data, files, and/or databases may be stored, retrieved, accessed, and/or transmitted in human-readable formats such as numeric, textual, graphic, or multimedia formats, further including various types of markup language, among other possible formats. Alternatively or in combination with the above formats, the data, files, and/or databases may be stored, retrieved, accessed, and/or transmitted in binary, encoded, compressed, and/or encrypted formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform and/or unique identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C#, Objective-C, Java, Scala, Clojure, Elixir, Swift, Go, Perl, PHP, Python, Ruby, JavaScript, Web Assembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some aspects, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), may cause such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 4. In particular, aspects may operate with software, hardware, and/or operating system implementations other than those described herein.

The imager 116 in FIG. 1 may include one or more optical systems. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being images.

In some instances, improvements in imaging performance, e.g., for dual-side (flow cell) imaging applications comprising the use of thick flow cell walls (e.g., wall (or coverslip) thickness>700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Exemplary aspects disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 μm, at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some aspects, the numerical aperture may be at least 0.75. In some aspects, the numerical aperture is at least 1.0. In some aspects, the working distance is at least 850 μm. In some aspects, the working distance is at least 1,000 μm. In some aspects, the field-of-view may have an area of at least 2.5 mm2. In some aspects, the field-of-view may have an area of at least 3 mm2. In some aspects, the spatial sampling frequency may be at least 2.5 times the optical resolution of the fluorescence imaging system. In some aspects, the spatial sampling frequency may be at least 3 times the optical resolution of the fluorescence imaging system. In some aspects, the system may further comprise an X-Y-Z translation stage such that the system is configured to acquire a series of two or more fluorescence images in an automated fashion, wherein each image of the series is or may be acquired for a different field-of-view. In some aspects, a position of the sample plane may be simultaneously adjusted in an X direction, a Y direction, and a Z direction to match the position of an objective lens focal plane in between acquiring images for different fields-of-view. In some aspects, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some aspects, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some aspects, the specified error threshold is 100 nm or greater. In some aspects, the specified error threshold is 50 nm or less. In some aspects, the system comprises three or more image sensors, and wherein the system is configured to image fluorescence in each of three or more wavelength ranges onto a different image sensor. In some aspects, a difference in the position of a focal plane for each of the three or more image sensors and the sample plane is less than 100 nm. In some aspects, a difference in the position of a focal plane for each of the three or more image sensors and the sample plane is less than 50 nm. In some aspects, the total time required to reposition the sample plane, adjust focus if necessary, and acquire an image is less than 0.4 seconds per field-of-view. In some aspects, the total time required to reposition the sample plane, adjust focus if necessary, and acquire an image is less than 0.3 seconds per field-of-view.

Also discloser herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some aspects, the objective lens may be a commercially-available microscope objective. In some aspects, the commercially-available microscope objective may have a numerical aperture of at least 0.3. In some aspects, the objective lens may have a working distance of at least 700 μm. In some aspects, the objective lens may be corrected to compensate for a cover slip thickness (or flow cell wall thickness) of 0.17 mm or of greater or lesser thickness than 0.17 mm. In some aspects, the optical system may be corrected to compensate for cover slip thickness, flow cell thickness, or distance between desired focal planes. In some aspects, said correction may be made by inserting a corrective optic, such as a lens or optical assembly into the light path of the optical system. In some aspects, said correction may be made without inserting a corrective optic, such as a lens or optical assembly into the light path of the optical system. In some aspects, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some aspects, the at least one tube lens may be a compound lens comprising three or more optical components. In some aspects, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some aspects, the at least one tube lens is configured to correct an imaging performance metric for a combination of the objective lens, the at least one tube lens, and the at least one image sensor when imaging an interior surface of a flow cell having a wall thickness of at least 1 mm. In some aspects, the at least one tube lens is configured to correct an imaging performance metric for a combination of the objective lens, the at least one tube lens, and the at least one image sensor when imaging an interior surface of a flow cell having a gap of at least 100 μm. In some aspects, the at least one tube lens is configured to correct an imaging performance metric for a combination of the objective lens, the at least one tube lens, and the at least one image sensor when imaging an interior surface of a flow cell having a gap of at least 200 μm. In some aspects, the system comprises a single objective lens, two tube lenses, and two image sensors, and each of the two tube lenses is designed to provide optimal imaging performance at a different fluorescence wavelength. In some aspects, the system comprises a single objective lens, three tube lenses, and three image sensors, and each of the three tube lenses is designed to provide optimal imaging performance at a different fluorescence wavelength. In some aspects, the system comprises a single objective lens, four tube lenses, and four image sensors, and each of the four tube lenses is designed to provide optimal imaging performance at a different fluorescence wavelength. In some aspects, the design of the objective lens or the at least one tube lens is configured to optimize the modulation transfer function in the mid to high spatial frequency range. In some aspects, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some aspects, the difference in the imaging performance metric for imaging the upper interior surface and the lower interior surface of the flow cell is less than 10%. In some aspects, the difference in imaging performance metric for imaging the upper interior surface and the lower interior surface of the flow cell is less than 5%. In some aspects, the use of the at least one tube lens provides for an at least equivalent or better improvement in the imaging performance metric for dual-side imaging compared to that for a conventional system comprising an objective lens, a motion-actuated compensator, and an image sensor. In some aspects, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some aspects, the illumination system further comprises a condenser lens. In some aspects, the specified field-of-illumination has an area of at least 2 mm². In some aspects, the light delivered to the specified field-of-illumination is of uniform intensity across a specified field-of-view for an imaging system used to acquire images of the support surface. In some aspects, the specified field-of-view has an area of at least 2 mm². In some aspects, the light delivered to the specified field-of-illumination is of uniform intensity across the specified field-of-view when a coefficient of variation (CV) for light intensity is less than 10%. In some aspects, the light delivered to the specified field-of-illumination is of uniform intensity across the specified field-of-view when a coefficient of variation (CV) for light intensity is less than 5%. In some aspects, the light delivered to the specified field-of-illumination has a speckle contrast value of less than 0.1. In some aspects, the light delivered to the specified field-of-illumination has a speckle contrast value of less than 0.05.

Imaging modules and systems: It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectric focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

Aspects of the present disclosure provide methods for sequencing immobilized or non-immobilized template molecules. The methods may be operated in system 100, for example, in sequencer 114. In some aspects, the immobilized template molecules comprise a plurality of nucleic acid template molecules having one copy of a target sequence of interest. In some aspects, nucleic acid template molecules having one copy of a target sequence of interest may be generated by conducting bridge amplification using linear library molecules. In some aspects, the immobilized template molecules comprise a plurality of nucleic acid template molecules each having two or more tandem copies of a target sequence of interest (e.g., concatemers). In some aspects, nucleic acid template molecules comprising concatemer molecules may be generated by conducting rolling circle amplification of circularized linear library molecules. In some aspects, the non-immobilized template molecules comprise circular molecules. In some aspects, methods for sequencing employ soluble (e.g., non-immobilized) sequencing polymerases or sequencing polymerases that are immobilized to a support.

In some aspects, the sequencing reactions employ detectably labeled nucleotide analogs. In some aspects, the sequencing reactions employ a two-stage sequencing reaction comprising binding detectably labeled multivalent molecules, and incorporating nucleotide analogs. In some aspects, the sequencing reactions employ non-labeled nucleotide analogs. In some aspects, the sequencing reactions employ phosphate chain labeled nucleotides.

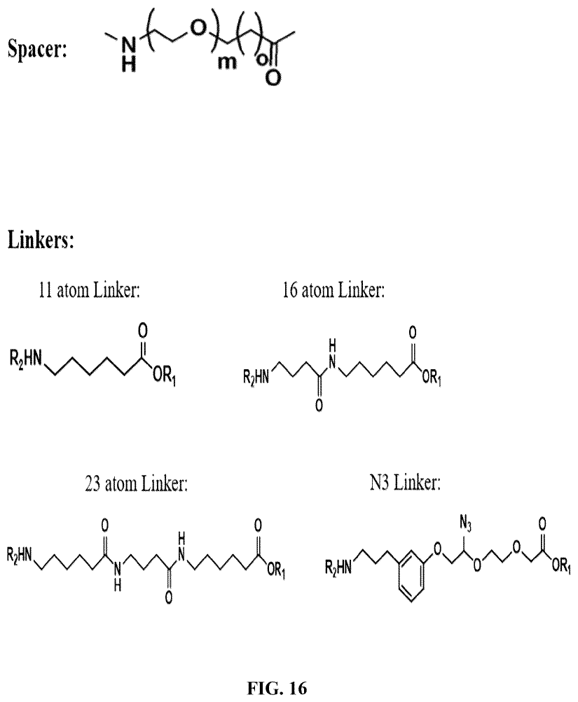

In some aspects, the immobilized concatemers each comprise tandem repeat units of the sequence-of-interest (e.g., insert region) and any adaptor sequences. For example, the tandem repeat unit comprises: (i) a left universal adaptor sequence having a binding sequence for a first surface primer (920) (e.g., surface pinning primer), (ii) a left universal adaptor sequence having a binding sequence for a first sequencing primer (940) (e.g., forward sequencing primer), (iii) a sequence-of-interest (910), (iv) a right universal adaptor sequence having a binding sequence for a second sequencing primer (950) (e.g., reverse sequencing primer), (v) a right universal adaptor sequence having a binding sequence for a second surface primer (930) (e.g., surface capture primer), and (vii) a left sample index sequence (960) and/or a right sample index sequence (970). In some aspects, the tandem repeat unit further comprises a left unique identification sequence (980) and/or a right unique identification sequence (990). In some aspects, the tandem repeat unit further comprises at least one binding sequence for a compaction oligonucleotide. In some aspects, FIGS. 15 and 16 show linear library molecules for a unit of a concatemer molecule.

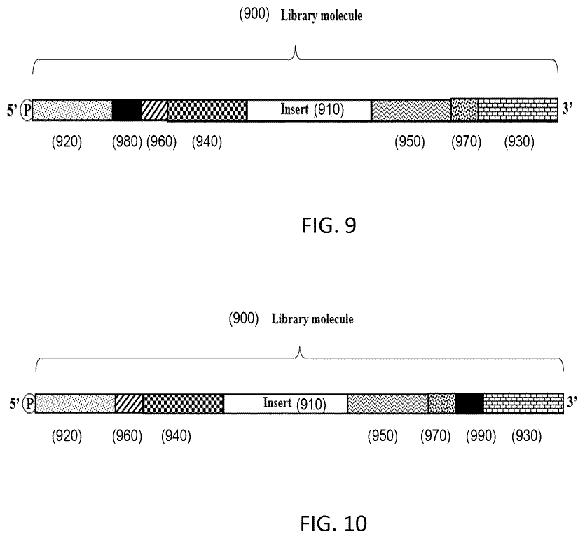

Specifically, FIG. 9 is a schematic showing an exemplary linear single stranded library molecule (900) which includes: a surface pinning primer binding site (920); an optional left unique identification sequence (980); a left index sequence (960); a forward sequencing primer binding site (940); an insert region having a sequence of interest (910); reverse sequencing primer binding site (950); a right index sequence (970); and a surface capture primer binding site (930).

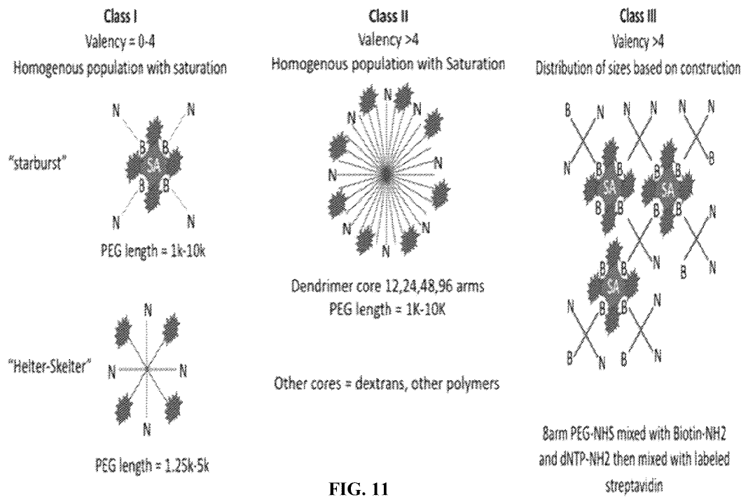

FIG. 10 is a schematic showing an exemplary linear single stranded library molecule (900) which includes: a surface pinning primer binding site (920); a left index sequence (960); a forward sequencing primer binding site (940); an insert region having a sequence of interest (910); a reverse sequencing primer binding site (950); a right index sequence (970); an optional right unique identification sequence (990); and a surface capture primer binding site (930).

FIG. 11 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

The immobilized concatemer may self-collapse into a compact nucleic acid nanoball. Inclusion of one or more compaction oligonucleotides during the RCA reaction may further compact the size and/or shape of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yields an increased signal intensity for each concatemer. Multiple portions of a given concatemer may be simultaneously sequenced. Furthermore, a plurality of binding complexes may form along a particular concatemer molecule, each binding complex comprising a sequencing polymerase bound to a template/primer duplex and bound to a multivalent molecule, wherein the plurality of binding complexes remain stable without dissociation resulting in increased persistence time which increases signal intensity and reduces imaging time.

Methods for Sequencing using Nucleotide Analogs

Aspects of the present disclosure provide methods for sequencing any of the immobilized template molecules described herein, the methods comprising step (a): contacting a sequencing polymerase to (i) a nucleic acid template molecule and (ii) a nucleic acid sequencing primer, wherein the contacting is conducted under a condition suitable to bind the sequencing polymerase to the nucleic acid template molecule which is hybridized to the nucleic acid primer, wherein the nucleic acid template molecule hybridized to the nucleic acid primer forms the nucleic acid duplex. In some aspects, the sequencing polymerase comprises a recombinant mutant sequencing polymerase that may bind and incorporate nucleotide analogs.

In some aspects, in the methods for sequencing template molecules, the sequencing primer comprises a 3′ extendible end or a 3′ non-extendible end. In some aspects, the plurality of nucleic acid template molecules comprise amplified template molecules (e.g., clonally amplified template molecules). In some aspects, the plurality of nucleic acid template molecules comprise one copy of a target sequence of interest. In some aspects, the plurality of nucleic acid molecules comprise two or more tandem copies of a target sequence of interest (e.g., concatemers). In some aspects, the plurality of nucleic acid template molecules comprise the same target sequence of interest or different target sequences of interest. In some aspects, the plurality of nucleic acid primers are in solution or are immobilized to a support. In some aspects, when the plurality of nucleic acid template molecules and/or the plurality of nucleic acid primers are immobilized to a support, the binding with the first sequencing polymerase generates a plurality of immobilized first complexed polymerases. In some aspects, the plurality of nucleic acid template molecules and/or nucleic acid primers are immobilized to 10²-10¹⁵ different sites on a support. In some aspects, the binding of the plurality of template molecules and nucleic acid primers with the plurality of first sequencing polymerases generates a plurality of first complexed polymerases immobilized to 10²-10¹⁵ different sites on the support. In some aspects, the plurality of immobilized first complexed polymerases on the support are immobilized to pre-determined or to random sites on the support. In some aspects, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some aspects, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some aspects, the sequencing polymerase is contacted with the plurality of nucleotides in the presence of at least one catalytic cation comprising magnesium and/or manganese. In some aspects, the plurality of nucleotides comprises at least one nucleotide analog having a chain terminating moiety at the sugar 2′ or 3′ position. In some aspects, the chain terminating moiety is removable from the sugar 2′ or 3′ position to convert the chain terminating moiety to an OH or H group. In some aspects, the plurality of nucleotides comprises at least one nucleotide that lacks a chain terminating moiety. In some aspects, at least on nucleotide is labeled with a detectable reporter moiety (e.g., fluorophore) that emits a detectable signal. The detectable reporter moiety comprises a fluorophore. In some aspects, the fluorophore is attached to the nucleo-base. In some aspects, the fluorophore is attached to the nucleo-base with a linker which is cleavable/removable from the base. In some aspects, at least one of the nucleotides in the plurality is not labeled with a detectable reporter moiety. In some aspects, a particular detectable reporter moiety (e.g., fluorophore) that is attached to the nucleotide may correspond to the nucleotide base (e.g., dATP, dGTP, dCTP, dTTP or dUTP) to permit detection and identification of the nucleo-base. When the incorporated chain terminating nucleotide is detectably labeled, step (b) further comprises detecting the emitted signal from the incorporated chain terminating nucleotide. In some aspects, step (b) further comprises identifying the nucleo-based of the incorporated chain terminating nucleotide.

In some aspects, the methods for sequencing further comprise step (c): removing the chain terminating moiety from the incorporated chain terminating nucleotide to generate an extendible 3′OH group. In some aspects, step (c) further comprises removing the detectable label from the incorporated chain terminating nucleotide. In some aspects, the sequencing polymerase remains bound to the template molecule which is hybridized to the sequencing primer which is extended by one nucleo-base.

In some aspects, the methods for sequencing further comprise step (d): repeating steps (b) and (c) at least once.

### Two-Stage Methods for Nucleic Acid Sequencing

Aspects of the present disclosure provide a two-stage method for sequencing any of the immobilized template molecules described herein. In some aspects, the first stage generally comprises binding multivalent molecules to complexed polymerases to form multivalent-complexed polymerases, and detecting the multivalent-complexed polymerases.

In some aspects, the first stage comprises step (a): contacting a plurality of a first sequencing polymerase to (i) a plurality of nucleic acid template molecules and (ii) a plurality of nucleic acid sequencing primers, wherein the contacting is conducted under a condition suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex wherein the nucleic acid duplex comprises a nucleic acid template molecule hybridized to a nucleic acid primer. In some aspects, the first polymerase comprises a recombinant mutant sequencing polymerase.

In some aspects, in the methods for sequencing template molecules, the sequencing primer comprises an oligonucleotide having a 3′ extendible end or a 3′ non-extendible end. In some aspects, the plurality of nucleic acid template molecules comprise amplified template molecules (e.g., clonally amplified template molecules). In some aspects, the plurality of nucleic acid template molecules comprise one copy of a target sequence of interest. In some aspects, the plurality of nucleic acid molecules comprise two or more tandem copies of a target sequence of interest (e.g., concatemers). In some aspects, the nucleic acid template molecules in the plurality of nucleic acid template molecules comprise the same target sequence of interest or different target sequences of interest. In some aspects, the plurality of nucleic acid template molecules and/or the plurality of nucleic acid primers are in solution or are immobilized to a support. In some aspects, when the plurality of nucleic acid template molecules and/or the plurality of nucleic acid primers are immobilized to a support, the binding with the first sequencing polymerase generates a plurality of immobilized first complexed polymerases. In some aspects, the plurality of nucleic acid template molecules and/or nucleic acid primers are immobilized to 10²-10¹⁵ different sites on a support. In some aspects, the binding of the plurality of template molecules and nucleic acid primers with the plurality of first sequencing polymerases generates a plurality of first complexed polymerases immobilized to 10²-10¹⁵ different sites on the support. In some aspects, the plurality of immobilized first complexed polymerases on the support are immobilized to pre-determined or to random sites on the support. In some aspects, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some aspects, the methods for sequencing further comprise step (b): contacting the plurality of first complexed polymerases with a plurality of multivalent molecules to form a plurality of multivalent-complexed polymerases (e.g., binding complexes). In some aspects, individual multivalent molecules in the plurality of multivalent molecules comprise a core attached to multiple nucleotide arms and each nucleotide arm is attached to a nucleotide (e.g., nucleotide unit) (e.g., FIGS. 11-15). In some aspects, the contacting of step (b) is conducted under a condition suitable for binding complementary nucleotide units of the multivalent molecules to at least two of the plurality of first complexed polymerases thereby forming a plurality of multivalent-complexed polymerases. In some aspects, the condition is suitable for inhibiting polymerase-catalyzed incorporation of the complementary nucleotide units into the primers of the plurality of multivalent-complexed polymerases. In some aspects, the plurality of multivalent molecules comprise at least one multivalent molecule having multiple nucleotide arms (e.g., FIGS. 11-14) each attached with a nucleotide analog (e.g., nucleotide analog unit), where the nucleotide analog includes a chain terminating moiety at the sugar 2′ and/or 3′ position. In some aspects, the plurality of multivalent molecules comprises at least one multivalent molecule comprising multiple nucleotide arms each attached with a nucleotide unit that lacks a chain terminating moiety. In some aspects, at least one of the multivalent molecules in the plurality of multivalent molecules is labeled with a detectable reporter moiety that emits a signal. In some aspects, the detectable reporter moiety comprises a fluorophore. In some aspects, the contacting of step (b) is conducted in the presence of at least one non-catalytic cation comprising strontium, barium and/or calcium.

In some aspects, the methods for sequencing further comprise step (c): detecting the plurality of multivalent-complexed polymerases. In some aspects, the detecting includes detecting the signals emitted by the multivalent molecules that are bound to the complexed polymerases, where the complementary nucleotide units of the multivalent molecules are bound to the primers but incorporation of the complementary nucleotide units is inhibited. In some aspects, the multivalent molecules are labeled with a detectable reporter moiety to permit detection. In some aspects, the labeled multivalent molecules comprise a fluorophore attached to the core, linker and/or nucleotide unit of the multivalent molecules.

In some aspects, the methods for sequencing further comprise step (d): identifying the nucleo-base of the complementary nucleotide units that are bound to the plurality of first complexed polymerases, thereby determining the sequence of the template molecule. In some aspects, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some aspects, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some aspects, the second stage of the two-stage sequencing method generally comprises nucleotide incorporation. In some aspects, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some aspects, the second sequencing polymerase comprises a recombinant mutant sequencing polymerase.

In some aspects, the plurality of first sequencing polymerases of step (a) have an amino acid sequence that is 100% identical to the amino acid sequence as the plurality of the second sequencing polymerases of step (f). In some aspects, the plurality of first sequencing polymerases of step (a) have an amino acid sequence that differs from the amino acid sequence of the plurality of the second sequencing polymerases of step (f).

In some aspects, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some aspects, the contacting of step (g) is conducted under a condition that is suitable for promoting polymerase-catalyzed incorporation of the bound complementary nucleotides into the primers of the nucleotide-complexed polymerases thereby extending the sequencing primer by one nucleo-base. In some aspects, the incorporating the nucleotide into the 3′ end of the sequencing primer in step (g) comprises a primer extension reaction. In some aspects, the contacting of step (g) is conducted in the presence of at least one catalytic cation comprising magnesium and/or manganese. In some aspects, the plurality of nucleotides comprise native nucleotides (e.g., non-analog nucleotides) or nucleotide analogs. In some aspects, the plurality of nucleotides comprise a 2′ and/or 3′ chain terminating moiety which is removable or is not removable. In some aspects, at least one of the nucleotides in the plurality is not labeled with a detectable reporter moiety. In some aspects, the plurality of nucleotides are non-labeled. In some aspects, the plurality of nucleotides comprises a plurality of nucleotides labeled with detectable reporter moiety. The detectable reporter moiety comprises a fluorophore. In some aspects, the fluorophore is attached to the nucleotide base. In some aspects, the fluorophore is attached to the nucleotide base with a linker which is cleavable/removable from the base or is not removable from the base. In some aspects, a particular detectable reporter moiety (e.g., fluorophore) that is attached to the nucleotide may correspond to the nucleotide base (e.g., dATP, dGTP, dCTP, dTTP or dUTP) to permit detection and identification of the nucleotide base.

In some aspects, when the plurality of nucleotides in step (g) are detectably labeled, the methods for sequencing further comprise step (h): detecting the complementary nucleotides which are incorporated into the primers of the nucleotide-complexed polymerases. In some aspects, the plurality of nucleotides are labeled with a detectable reporter moiety to permit detection. In some aspects, when the plurality of nucleotides in step (g) are non-labeled, the detecting of step (h) is omitted.

In some aspects, when the plurality of nucleotides in step (g) are detectably labeled, the methods for sequencing further comprise step (i): identifying the bases of the complementary nucleotides which are incorporated into the primers of the nucleotide-complexed polymerases. In some aspects, the identification of the incorporated complementary nucleotides in step (i) may be used to confirm the identity of the complementary nucleotides of the multivalent molecules that are bound to the plurality of first complexed polymerases in step (d). In some aspects, the identifying of step (i) may be used to determine the sequence of the nucleic acid template molecules. In some aspects, when the plurality of nucleotides in step (g) are non-labeled, the identifying of step (i) is omitted.

In some aspects, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some aspects, the methods for sequencing further comprise step (k): repeating steps (a)-(j) at least once. In some aspects, the sequence of the nucleic acid template molecules may be determined by detecting and identifying the multivalent molecules that bind the sequencing polymerases but do not incorporate into the 3′ end of the primer at steps (c) and (d). In some aspects, the sequence of the nucleic acid template molecule may be determined (or confirmed) by detecting and identifying the nucleotide that incorporates into the 3′ end of the primer at steps (h) and (i).

In some aspects, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some aspects, the first sequencing polymerase comprises any wild type or mutant polymerase described herein. In some aspects, the second sequencing polymerase comprises any wild type or mutant polymerase described herein. The concatemer template molecule comprises tandem repeat sequences of a sequence of interest and at least one universal sequencing primer binding site. The first and second nucleic acid primers may bind to a sequencing primer binding site along the concatemer template molecule. Exemplary multivalent molecules are shown in FIGS. 11-14.

In some aspects, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some aspects, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers may bind to a sequencing primer binding site along the concatemer template molecule. Exemplary multivalent molecules are shown in FIGS. 11-14.

### Sequencing-by-Binding

Aspects of the present disclosure provide methods for sequencing any of the immobilized template molecules described herein, wherein the sequencing methods comprise a sequencing-by-binding (SBB) procedure which employs non-labeled chain-terminating nucleotides. In some aspects, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

Methods for Sequencing using Phosphate-Chain Labeled Nucleotides

Aspects of the present disclosure provide methods for sequencing using immobilized sequencing polymerases which bind non-immobilized template molecules, wherein the sequencing reactions are conducted with phosphate-chain labeled nucleotides. In some aspects, the sequencing methods comprise step (a): providing a support having a plurality of sequencing polymerases immobilized thereon. In some aspects, the sequencing polymerase comprises a processive DNA polymerase. In some aspects, the sequencing polymerase comprises a wild type or mutant DNA polymerase, including for example a Phi29 DNA polymerase. In some aspects, the support comprise a plurality of separate compartments and a sequencing polymerase is immobilized to the bottom of a compartment. In some aspects, the separate compartments comprise a silica bottom through which light may penetrate. In some aspects, the separate compartments comprise a silica bottom configured with a nanophotonic confinement structure comprising a hole in a metal cladding film (e.g., aluminum cladding film). In some aspects, the hole in the metal cladding has a small aperture, for example, approximately 70 nm. In some aspects, the height of the nanophotonic confinement structure is approximately 100 nm. In some aspects, the nanophotonic confinement structure comprises a zero mode waveguide (ZMW). In some aspects, the nanophotonic confinement structure contains a liquid.

In some aspects, the sequencing method further comprises step (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some aspects, the individual sequencing primers hybridize to a universal sequencing primer binding site on the single stranded circular template molecule.

In some aspects, the sequencing method further comprises step (c): contacting the plurality of polymerase/template/primer complexes with a plurality of phosphate chain labeled nucleotides each comprising an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups may be referred to as alpha, beta and gamma phosphate groups. In some aspects, a particular detectable reporter moiety which is attached to the terminal phosphate group corresponds to the nucleotide base (e.g., dATP, dGTP, dCTP, dTTP or dUTP) to permit detection and identification of the nucleo-base. In some aspects, the plurality of polymerase/template/primer complexes are contacted with the plurality of phosphate chain labeled nucleotides under a condition suitable for polymerase-catalyzed nucleotide incorporation. In some aspects, the sequencing polymerases are capable of binding a complementary phosphate chain labeled nucleotide and incorporating the complementary nucleotide opposite a nucleotide in a template molecule. In some aspect, the polymerase-catalyzed nucleotide incorporation reaction cleaves between the alpha and beta phosphate groups thereby releasing a multi-phosphate chain linked to a fluorophore.

In some aspects, the sequencing method further comprises step (d): detecting the fluorescent signal emitted by the phosphate chain labeled nucleotide that is bound by the sequencing polymerase, and incorporated into the terminal end of the sequencing primer. In some aspects, step (d) further comprises identifying the phosphate chain labeled nucleotide that is bound by the sequencing polymerase, and incorporated into the terminal end of the sequencing primer.

In some aspects, the sequencing method further comprises step (d): repeating steps (c)-(d) at least once. In some aspects, sequencing methods that employ phosphate chain labeled nucleotides may be conducted according to the methods described in U.S. Pat. Nos. 7,170,050; 7,302,146; and/or 7,405,281.

### Sequencing Polymerases

Aspects of the present disclosure provide methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules. In some aspects, the sequencing polymerase(s) is/are capable of incorporating a complementary nucleotide opposite a nucleotide in a template molecule. In some aspects, the sequencing polymerase(s) is/are capable of binding a complementary nucleotide unit of a multivalent molecule opposite a nucleotide in a template molecule. In some aspects, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Nucleotides

Aspects of the present disclosure provide methods for sequencing nucleic acid molecules, where any of the sequencing methods described herein employ at least one nucleotide. The nucleotides comprise a base, sugar and at least one phosphate group. In some aspects, at least one nucleotide in the plurality comprises an aromatic base, a five carbon sugar (e.g., ribose or deoxyribose), and one or more phosphate groups (e.g., 1-10 phosphate groups). The plurality of nucleotides may comprise at least one type of nucleotide selected from a group consisting of dATP, dGTP, dCTP, dTTP and dUTP. The plurality of nucleotides may comprise at a mixture of any combination of two or more types of nucleotides selected from a group consisting of dATP, dGTP, dCTP, dTTP and/or dUTP. In some aspects, at least one nucleotide in the plurality is not a nucleotide analog. In some aspects, at least one nucleotide in the plurality comprises a nucleotide analog.

In some aspects, in any of the methods for sequencing nucleic acid molecules described herein, at least one nucleotide in the plurality of nucleotides comprise a chain of one, two or three phosphorus atoms where the chain is typically attached to the 5′ carbon of the sugar moiety via an ester or phosphoramide linkage. In some aspects, at least one nucleotide in the plurality is an analog having a phosphorus chain in which the phosphorus atoms are linked together with intervening O, S, NH, methylene or ethylene. In some aspects, the phosphorus atoms in the chain include substituted side groups including O, S or BH₃. In some aspects, the chain includes phosphate groups substituted with analogs including phosphoramidate, phosphorothioate, phosphordithioate, and O-methylphosphoroamidite groups.

In some aspects, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some aspects, the chain terminating moiety may inhibit polymerase-catalyzed incorporation of a subsequent nucleotide unit or free nucleotide in a nascent strand during a primer extension reaction. In some aspects, the chain terminating moiety is attached to 3′ sugar position where the sugar comprises a ribose or deoxyribose sugar moiety. In some aspects, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some aspects, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some aspects, the chain terminating moiety is cleavable/removable from the nucleotide, for example by reacting the chain terminating moiety with a chemical agent, pH change, light or heat. In some aspects, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some aspects, the chain terminating moieties aryl and benzyl are cleavable with H2 Pd/C. In some aspects, the chain terminating moieties amine, amide, keto, isocyanate, phosphate, thio, disulfide are cleavable with phosphine or with a thiol group including beta-mercaptoethanol or dithiothritol (DTT). In some aspects, the chain terminating moiety carbonate is cleavable with potassium carbonate (K₂CO₃) in MeOH, with triethylamine in pyridine, or with Zn in acetic acid (AcOH). In some aspects, the chain terminating moieties urea and silyl are cleavable with tetrabutylammonium fluoride, pyridine-HF, with ammonium fluoride, or with triethylamine trihydrofluoride. In some aspects, the chain terminating moiety may be cleavable/removable with nitrous acid. In some aspects, a chain terminating moiety may be cleavable/removable using a solution comprising nitrite, such as, for example, a combination of nitrite with an acid such as acetic acid, sulfuric acid, or nitric acid. In some further aspects, said solution may comprise an organic acid.

In some aspects, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some aspects, the chain terminating moiety comprises an azide, azido or azidomethyl group. In some aspects, the chain terminating moiety comprises a 3′-O-azido or 3′-O-azidomethyl group. In some aspects, the chain terminating moieties azide, azido and azidomethyl group are cleavable/removable with a phosphine compound. In some aspects, the phosphine compound comprises a derivatized tri-alkyl phosphine moiety or a derivatized tri-aryl phosphine moiety. In some aspects, the phosphine compound comprises Tris(2-carboxyethyl) phosphine (TCEP) or bis-sulfo triphenyl phosphine (BS-TPP) or Tri (hydroxyproyl)phosphine (THPP). In some aspects, the cleaving agent comprises 4-dimethylaminopyridine (4-DMAP). In some aspects, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some aspects, the chain terminating moiety comprising one or more of a 3′-O-amino group, a 3′-O-aminomethyl group, a 3′-O-methylamino group, or derivatives thereof may be cleaved using a solution comprising nitrite. In some aspects, for example, nitrite may be combined with or contacted with an acid such as acetic acid, sulfuric acid, or nitric acid. In some further aspects, for example, nitrite may be combined with or contacted with an organic acid such as, for example, formic acid, acetic acid, propionic acid, butyric acid, isobutyric acid, or the like. In some aspects, the chain terminating moiety comprises a 3′-acetal moiety which may be cleaved with a palladium deblocking reagent (e.g., Pd(0)).

In some aspects, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety or derivatives thereof.

In some aspects, in any of the methods for sequencing nucleic acid molecules described herein, the plurality of nucleotides comprises a plurality of nucleotides labeled with detectable reporter moiety. The detectable reporter moiety comprises a fluorophore. In some aspects, the fluorophore is attached to the nucleotide base. In some aspects, the fluorophore is attached to the nucleotide base with a linker which is cleavable/removable from the base. In some aspects, at least one of the nucleotides in the plurality is not labeled with a detectable reporter moiety. In some aspects, a particular detectable reporter moiety (e.g., fluorophore) that is attached to the nucleotide may correspond to the nucleotide base (e.g., dATP, dGTP, dCTP, dTTP or dUTP) to permit detection and identification of the nucleotide base.

In some aspects, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some aspects, the cleavable linker on the base is cleavable/removable from the base by reacting the cleavable moiety with a chemical agent, pH change, light or heat. In some aspects, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some aspects, the cleavable moieties aryl and benzyl are cleavable with H2 Pd/C. In some aspects, the cleavable moieties amine, amide, keto, isocyanate, phosphate, thio, disulfide are cleavable with phosphine or with a thiol group including beta-mercaptoethanol or dithiothritol (DTT). In some aspects, the cleavable moiety carbonate is cleavable with potassium carbonate (K₂CO₃) in MeOH, with triethylamine in pyridine, or with Zn in acetic acid (AcOH). In some aspects, the cleavable moieties urea and silyl are cleavable with tetrabutylammonium fluoride, pyridine-HF, with ammonium fluoride, or with triethylamine trihydrofluoride.

In some aspects, in any of the methods for sequencing nucleic acid molecules described herein, the cleavable linker on the nucleotide base comprises cleavable moiety including an azide, azido or azidomethyl group. In some aspects, the cleavable moieties azide, azido and azidomethyl group are cleavable/removable with a phosphine compound. In some aspects, the phosphine compound comprises a derivatized tri-alkyl phosphine moiety or a derivatized tri-aryl phosphine moiety. In some aspects, the phosphine compound comprises Tris(2-carboxyethyl)phosphine (TCEP) or bis-sulfo triphenyl phosphine (BS-TPP) or Tri(hydroxyproyl)phosphine (THPP). In some aspects, the cleaving agent comprises 4-dimethylaminopyridine (4-DMAP).

In some aspects, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some aspects, the chain terminating moiety (e.g., at the sugar 2′ and/or sugar 3′ position) and the detectable reporter moiety linked to the base are chemically cleavable/removable with the same chemical agent. In some aspects, the chain terminating moiety (e.g., at the sugar 2′ and/or sugar 3′ position) and the detectable reporter moiety linked to the base are chemically cleavable/removable with different chemical agents.

### Multivalent Molecules

Aspects of the present disclosure provide methods for sequencing nucleic acid molecules, where any of the sequencing methods described herein employ at least one multivalent molecule. In some aspects, the multivalent molecule comprises a plurality of nucleotide arms attached to a core and having any configuration including a starburst, helter skelter, or bottle brush configuration (e.g., FIG. 11). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some aspects, the nucleotide unit comprises a base, sugar and at least one phosphate group, and the linker is attached to the nucleotide unit through the base. In some aspects, the linker comprises an aliphatic chain or an oligo ethylene glycol chain where both linker chains having 2-6 subunits. In some aspects, the linker also includes an aromatic moiety.

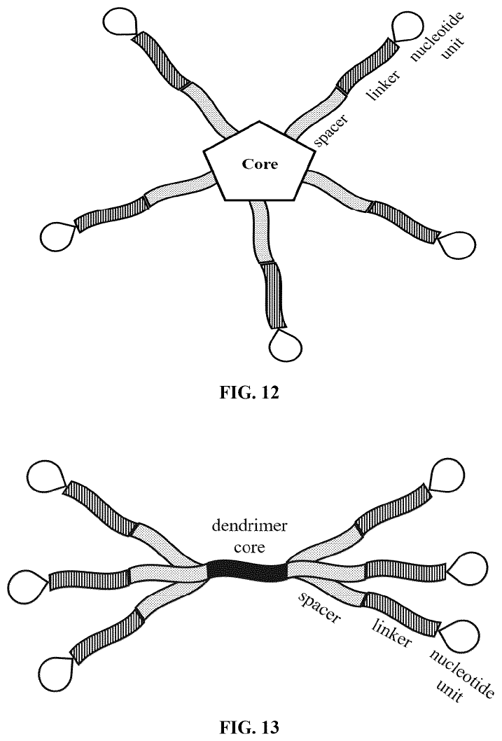

FIG. 12 is a schematic of an exemplary multivalent molecule comprising a generic core attached to a plurality of nucleotide-arms.

FIG. 13 is a schematic of an exemplary multivalent molecule comprising a dendrimer core attached to a plurality of nucleotide-arms.

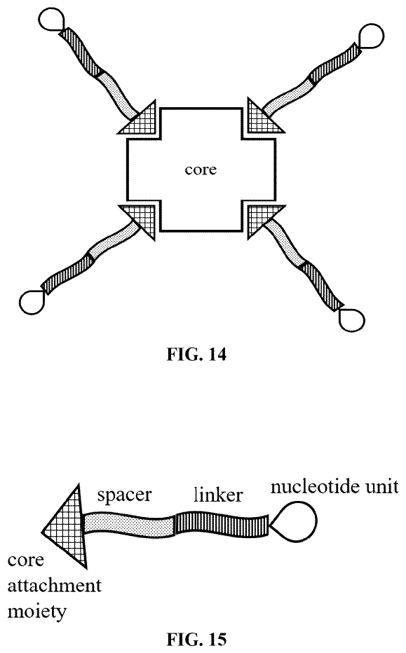

FIG. 14 shows a schematic of an exemplary multivalent molecule comprising a core attached to a plurality of nucleotide-arms, where the nucleotide arms comprise biotin, spacer, linker and a nucleotide unit.

FIG. 15 is a schematic of an exemplary nucleotide-arm comprising a core attachment moiety, spacer, linker and nucleotide unit.

FIG. 16 shows the chemical structure of an exemplary spacer (top), and the chemical structures of various exemplary linkers, including an 11-atom Linker, 16-atom Linker, 23-atom Linker and an N3 Linker (bottom).

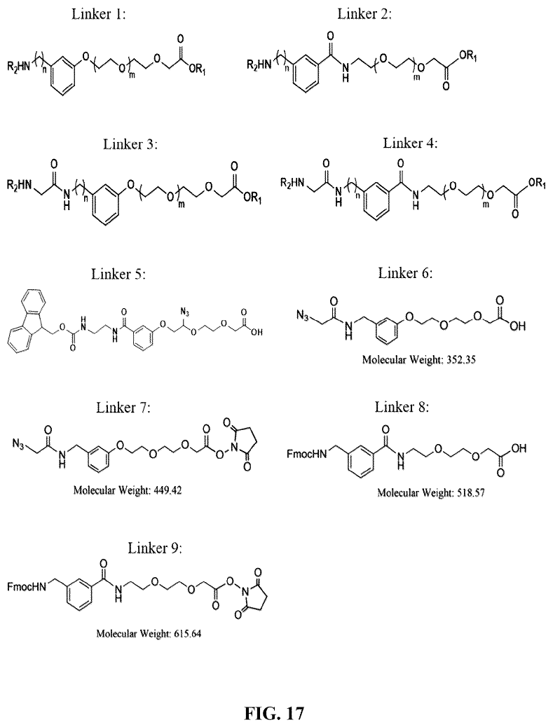

FIG. 17 shows the chemical structures of various exemplary linkers, including Linkers 1-9.

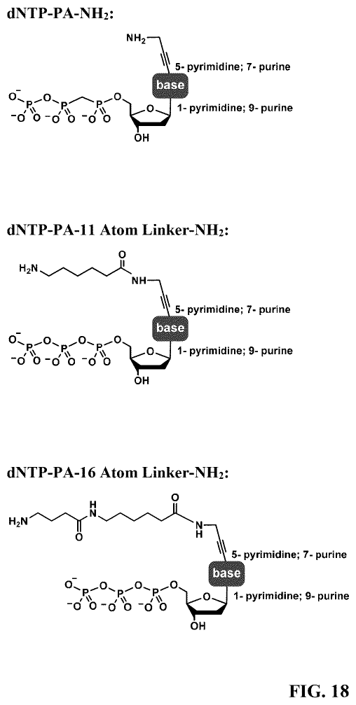

FIG. 18 shows the chemical structures of various exemplary linkers joined/attached to nucleotide units.

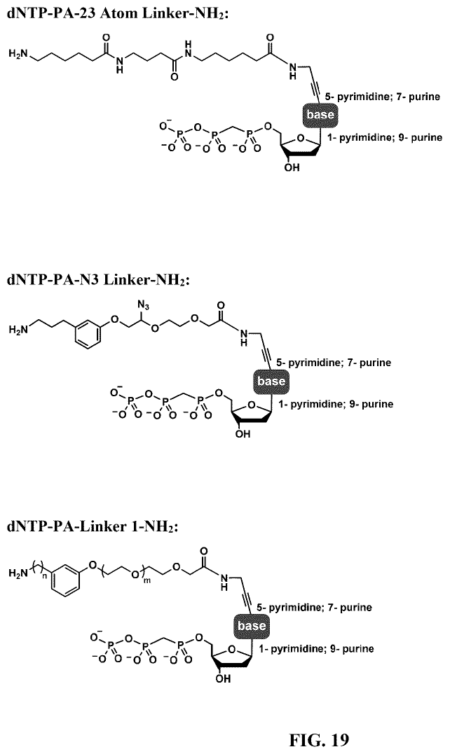

FIG. 19 shows the chemical structures of various exemplary linkers joined/attached to nucleotide units.

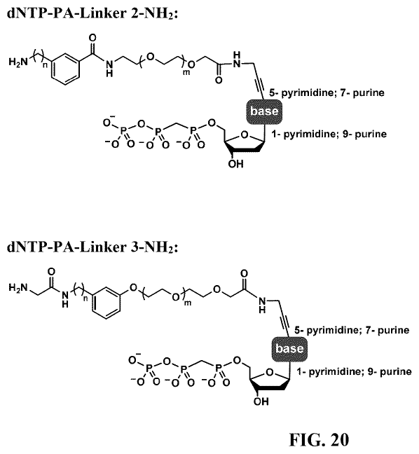

FIG. 20 shows the chemical structures of various exemplary linkers joined/attached to nucleotide units.

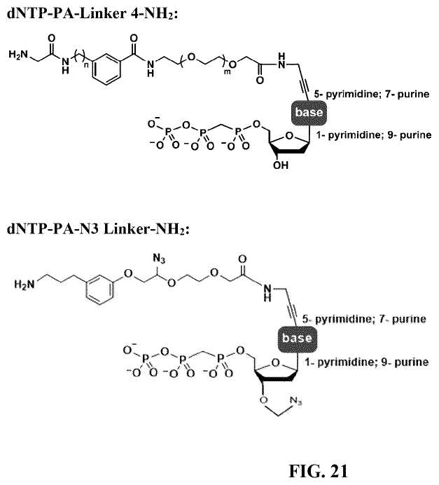

FIG. 21 shows the chemical structures of various exemplary linkers joined/attached to nucleotide units.

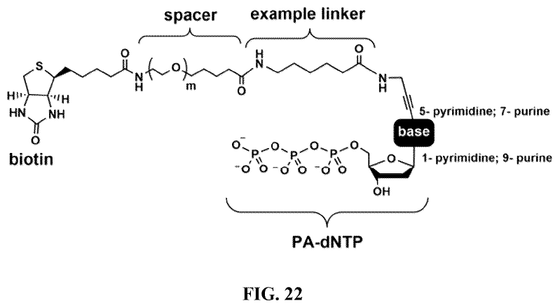

FIG. 22 shows the chemical structure of an exemplary biotinylated nucleotide-arm. In this exemplary example, the nucleotide unit is connected to the linker via a propargyl amine attachment at the 5 position of a pyrimidine base or the 7 position of a purine base.

In some aspects, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some aspects, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules may comprise one type multivalent molecule having one type of nucleotide unit selected from a group consisting of dATP, dGTP, dCTP, dTTP and dUTP. The plurality of multivalent molecules may comprise at a mixture of any combination of two or more types of multivalent molecules, where individual multivalent molecules in the mixture comprise nucleotide units selected from a group consisting of dATP, dGTP, dCTP, dTTP and/or dUTP.

In some aspects, the nucleotide unit comprises a chain of one, two or three phosphorus atoms where the chain is typically attached to the 5′ carbon of the sugar moiety via an ester or phosphoramide linkage. In some aspects, at least one nucleotide unit is a nucleotide analog having a phosphorus chain in which the phosphorus atoms are linked together with intervening O, S, NH, methylene or ethylene. In some aspects, the phosphorus atoms in the chain include substituted side groups including O, S or BH₃. In some aspects, the chain includes phosphate groups substituted with analogs including phosphoramidate, phosphorothioate, phosphordithioate, and O-methylphosphoroamidite groups.

In some aspects, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some aspects, the nucleotide unit comprises a chain terminating moiety (e.g., blocking moiety) at the sugar 2′ position, at the sugar 3′ position, or at the sugar 2′ and 3′ position. In some aspects, the chain terminating moiety may inhibit polymerase-catalyzed incorporation of a subsequent nucleotide unit or free nucleotide in a nascent strand during a primer extension reaction. In some aspects, the chain terminating moiety is attached to 3′ sugar position where the sugar comprises a ribose or deoxyribose sugar moiety. In some aspects, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some aspects, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some aspects, the chain terminating moiety is cleavable/removable from the nucleotide unit, for example by reacting the chain terminating moiety with a chemical agent, pH change, light or heat. In some aspects, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some aspects, the chain terminating moieties aryl and benzyl are cleavable with H2 Pd/C. In some aspects, the chain terminating moieties amine, amide, keto, isocyanate, phosphate, thio, disulfide are cleavable with phosphine or with a thiol group including beta-mercaptoethanol or dithiothritol (DTT). In some aspects, the chain terminating moiety carbonate is cleavable with potassium carbonate (K₂CO₃) in MeOH, with triethylamine in pyridine, or with Zn in acetic acid (AcOH). In some aspects, the chain terminating moieties urea and silyl are cleavable with tetrabutylammonium fluoride, pyridine-HF, with ammonium fluoride, or with triethylamine trihydrofluoride.

In some aspects, the nucleotide unit comprises a chain terminating moiety (e.g., blocking moiety) at the sugar 2′ position, at the sugar 3′ position, or at the sugar 2′ and 3′ position. In some aspects, the chain terminating moiety comprises an azide, azido or azidomethyl group. In some aspects, the chain terminating moiety comprises a 3′-O-azido or 3′-O-azidomethyl group. In some aspects, the chain terminating moieties azide, azido and azidomethyl group are cleavable/removable with a phosphine compound. In some aspects, the phosphine compound comprises a derivatized tri-alkyl phosphine moiety or a derivatized tri-aryl phosphine moiety. In some aspects, the phosphine compound comprises Tris(2-carboxyethyl) phosphine (TCEP) or bis-sulfo triphenyl phosphine (BS-TPP) or Tri(hydroxyproyl)phosphine (THPP). In some aspects, the cleaving agent comprises 4-dimethylaminopyridine (4-DMAP).

In some aspects, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some aspects, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some aspects, the detectable reporter moiety comprises a fluorophore. In some aspects, a particular detectable reporter moiety (e.g., fluorophore) that is attached to the multivalent molecule may correspond to the base (e.g., dATP, dGTP, dCTP, dTTP or dUTP) of the nucleotide unit to permit detection and identification of the nucleotide base.

In some aspects, at least one nucleotide arm of a multivalent molecule has a nucleotide unit that is attached to a detectable reporter moiety. In some aspects, the detectable reporter moiety is attached to the nucleotide base. In some aspects, the detectable reporter moiety comprises a fluorophore. In some aspects, a particular detectable reporter moiety (e.g., fluorophore) that is attached to the multivalent molecule may correspond to the base (e.g., dATP, dGTP, dCTP, dTTP or dUTP) of the nucleotide unit to permit detection and identification of the nucleotide base.

In some aspects, the core of a multivalent molecule comprises an avidin-like or streptavidin-like moiety and the core attachment moiety comprises biotin. In some aspects, the core comprises an streptavidin-type or avidin-type moiety which includes an avidin protein, as well as any derivatives, analogs and other non-native forms of avidin that may bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRA VIDIN and NEUTRALITE AVIDIN.

In some aspects, any of the methods for sequencing nucleic acid molecules described herein may include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some aspects, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some aspects, the present disclosure provides said method wherein the binding complex is deposited on, attached to, or hybridized to, a surface showing a contrast to noise ratio in the detecting step of greater than 20. In some aspects, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

A compaction oligonucleotide comprises a single-stranded linear oligonucleotide having a 5′ region that may hybridize to a first portion of a concatemer molecule and the compaction oligonucleotide having a 3′ region that may hybridize to a second portion of the concatemer molecule (e.g., the same concatemer molecule). In some aspects, hybridization of the compaction oligonucleotides to individual concatemer molecules causes the concatemer molecule to collapse or fold into a DNA nanoball which is more compact in shape and size compared to a non-collapsed DNA molecule. A spot image of a DNA nanoball may be represented as a Gaussian spot and the size may be measured as a full width half maximum (FWHM). A smaller spot size as indicated by a smaller FWHM typically correlates with an improved image of the spot. In some aspects, the FWHM of a DNA nanoball spot may be about 10 μm or smaller. The DNA nanoball may be a compact nucleic acid structure having a full width half maximum (FWHM) that is smaller compared to a concatemer that is not collapsed/folded into a DNA nanoball.

In some aspects, compaction oligonucleotides comprise a single stranded oligonucleotides comprising DNA, RNA, or a combination of DNA and RNA. The compaction oligonucleotides may be any length, including 20-150 nucleotides, or 30-100 nucleotides, or 40-80 nucleotides in length.

In some aspects, the compaction oligonucleotides comprises a 5′ region and a 3′ region, and optionally an intervening region between 5′ and 3′ regions. The intervening region may be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides may be wholly complementary or partially complementary along its length to a first portion of a concatemer molecule. The 3′ region of the compaction oligonucleotides may be wholly complementary or partially complementary along its length to a second portion of a concatemer molecule. The 5′ region of the compaction oligonucleotides may hybridize to a first universal sequence portion of a concatemer molecule. The 3′ region of the compaction oligonucleotides may hybridize to a second universal sequence portion of a concatemer molecule. The 5′ and 3′ regions of the compaction oligonucleotide may hybridize to the concatemer to pull together distal portions of the concatemer causing compaction of the concatemer to form a DNA nanoball.

The 5′ region of the compaction oligonucleotide may have the same sequence as the 3′ region. The 5′ region of the compaction oligonucleotide may have a sequence that is different from 3′ region. The 3′ region of the compaction oligonucleotide may have a sequence that is a reverse sequence of 5′ region.

In some aspects sequence data may be derived through nanopore sequencing, which comprises sequencing of a nucleic acid by translocating said nucleic acid across a membrane, such as through a pore, and wherein sequence reads or base calls are made by measuring one or more signals during the translocation event, such as impedance, current, voltage, or capacitance. In some aspects, the identity of a nucleotide may be determined by distinctive electrical signatures, such as the timing, duration, extent, or lineshape of a current block, impedance change, voltage change, or capacitance change. Sequencing of nucleic acids by translocation across a membrane and/or through a pore does not foreclose alternative detection methods, such as optical, chemical, biochemical, fluorescent, luminescent, magnetic, electromagnetic, acoustic, or electroacoustic detection.

It is to be appreciated that the Detailed Description section, and not any other section, is intended to be used to interpret the claims. Other sections may set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative aspects may perform functional blocks, steps, operations, methods, etc. using orderings different from those described herein.

References herein to “one aspect,” “an aspect,” “an example aspect,” “some aspects,” or similar phrases, indicate that the aspect described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein.

Additionally, some aspects may be described using the expression “coupled” and “connected” along with their derivatives. These terms are not necessarily intended as synonyms for each other. For example, some aspects may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

### Supports and Low Non-Specific Coatings

In some aspects, the flow cell 112 in FIG. 1 may include a support, e.g., a solid support as disclosed herein. Aspects of the present disclosure provide pairwise sequencing compositions and methods which employ a support comprising a plurality of oligonucleotide surface primers immobilized thereon. In some aspects, the support is passivated with a low non-specific binding coating. The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

The low non-specific binding coating comprises one layer or multiple layers. In some aspects, the plurality of surface primers are immobilized to the low non-specific binding coating. In some aspects, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached surface primers that may be used for tethering single-stranded nucleic acid library molecules to the support. In some aspects, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some aspects, the support structure may comprise one or more surfaces within an integrated or assembled microfluidic flow cell. The support structure may comprise one or more surfaces within a microplate format, e.g., the bottom surface of the wells in a microplate. In some aspects, the support structure comprises the interior surface (such as the lumen surface) of a capillary. In some aspects, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some aspects, the first layer may be covalently attached to the surface. In some aspects, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some aspects, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some aspects, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface may include, but are not limited to, biotin, methoxy ether, carboxylate, amine, NHS ester, maleimide, and bis-silane. In some aspects, two or more layers of a hydrophilic polymer, e.g., a linear polymer, branched polymer, or multi-branched polymer, may be deposited on the surface. In some aspects, two or more layers may be covalently coupled to each other or internally cross-linked to improve the stability of the resulting coating. In some aspects, surface primers with different nucleotide sequences and/or base modifications (or other biomolecules, e.g., enzymes or antibodies) may be tethered to the resulting layer at various surface densities. In some aspects, for example, both surface functional group density and surface primer concentration may be varied to attain a desired surface primer density range. Additionally, surface primer density may be controlled by diluting the surface primers with other molecules that carry the same functional group. For example, amine-labeled surface primers may be diluted with amine-labeled polyethylene glycol in a reaction with an NHS-ester coated surface to reduce the final primer density. Surface primers with different lengths of linker between the hybridization region and the surface attachment functional group may also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some aspects, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some aspects, the functionalized polymer coating comprises a poly(N-(5-azidoacetamidylpentyl) acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” may be accomplished using traditional crosslinking approaches with any compatible polymer or monomer subunits such that a surface comprising two or more highly cross-linked layers may be built sequentially. Examples of suitable polymers include, but are not limited to, streptavidin, poly acrylamide, polyester, dextran, poly-lysine, and copolymers of poly-lysine and PEG. In some aspects, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some aspects, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some aspects, the surface of the support structure used for nucleic acid hybridization and amplification may be a solid, non-porous surface. In some aspects, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some aspects, the support structure comprises the interior surface (such as the lumen surface) of a capillary. In some aspects the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some aspects, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some aspects, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some aspects, the label may comprise a fluorescent label. In some aspects, the label may comprise a radioisotope. In some aspects, the label may comprise any other detectable label known to one of skill in the art. In some aspects, the degree of non-specific binding exhibited by a given support surface formulation may thus be assessed in terms of the number of non-specifically bound protein molecules (or nucleic acid molecules or other molecules) per unit area. In some aspects, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some aspects, the coated support was immersed in a buffer (e.g., 25 mM ACES, pH 7.4) while the image was acquired.

In some aspects, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some aspects, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some aspects, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some aspects, a static contact angle may be determined. In some aspects, an advancing or receding contact angle may be determined. In some aspects, the water contact angle for the hydrophilic, low-binding support surfaced disclosed herein may range from about 0 degrees to about 30 degrees. In some aspects, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some aspects, the hydrophilic surfaces disclosed herein facilitate reduced wash times for bioassays, often due to reduced nonspecific binding of biomolecules to the low-binding surfaces. In some aspects, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some aspects, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some aspects, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some aspects, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some aspects, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some aspects, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some aspects, 1 primer or adapter sequence may be tethered to at least one layer of the surface. In some aspects, at least 2, 3, 4, 5, 6, 7, 8, 9, 10, or more than 10 different primer or adapter sequences may be tethered to at least one layer of the surface.

In some aspects, the tethered adapter and/or primer sequences may range in length from about 10 nucleotides to about 100 nucleotides. In some aspects, the tethered adapter and/or primer sequences may be at least 10, at least 20, at least 30, at least 40, at least 50, at least 60, at least 70, at least 80, at least 90, or at least 100 nucleotides in length. In some aspects, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some aspects the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some aspects, the resultant surface density of primers (e.g., capture primers) on the low binding support surfaces of the present disclosure may range from about 100 primer molecules per μm² to about 100,000 primer molecules per μm². In some aspects, the resultant surface density of primers on the low binding support surfaces of the present disclosure may range from about 1,000 primer molecules per μm² to about 1,000,000 primer molecules per μm². In some aspects, the surface density of primers may be at least 1,000, at least 10,000, at least 100,000, or at least 1,000,000 molecules per μm². In some aspects, the surface density of primers may be at most 1,000,000, at most 100,000, at most 10,000, or at most 1,000 molecules per μm². Any of the lower and upper values described in this paragraph may be combined to form a range included within the present disclosure, for example, in some aspects the surface density of primers may range from about 10,000 molecules per μm² to about 100,000 molecules per μm². Those of skill in the art will recognize that the surface density of primer molecules may have any value within this range, e.g., about 455,000 molecules per μm². In some aspects, the surface density of target library nucleic acid sequences initially hybridized to adapter or primer sequences on the support surface may be less than or equal to that indicated for the surface density of tethered primers. In some aspects, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some aspects, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it may be shown that improved CNR may provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) may be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (Signal)-B (interstial) in the FOV) yields a discernable feature that may be classified. In some aspects, the intrastitial background (B (intrastitial)) may contribute a confounding fluorescence signal that is not specific to the target of interest, but is present in the same ROI thus making it far more difficult to average and subtract.

Nucleic acid amplification on the low-binding coated supports described herein may decrease the B (interstitial) background signal by reducing non-specific binding, may lead to improvements in specific nucleic acid amplification, and may lead to a decrease in non-specific amplification that may impact the background signal arising from both the interstitial and intrastitial regions. In some aspects, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

The headings provided herein are not limitations of the various aspects of the disclosure, which aspects may be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that may be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” may mean within one or more than one standard deviation per the practice in the art. Alternatively, “about” or “approximately” may mean a range of up to 10% (i.e., +10%) or more depending on the limitations of the measurement system. For example, about 5 mg may include any number between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges may include the endpoints of the ranges and/or subranges.

The term “polony” used herein refers to a nucleic acid library molecule may be clonally amplified in-solution or on-support to generate an amplicon that may serve as a template molecule for sequencing. In some aspects, a linear library molecule mayb e circularized to generate a circularized library molecule, and the circularized library molecule may be clonally amplified in-solution or on-support to generate a concatemer. In some aspects, the concatemer may serve as a nucleic acid template molecule which may be sequenced. The concatemer is sometimes referred to as a polony. In some aspects, a polony includes denatured, cloned nucleotide strands.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, comprises any enzyme that may catalyze polymerization of nucleotides (including analogs thereof) into a nucleic acid strand. Typically but not necessarily such nucleotide polymerization may occur in a template-dependent fashion. Typically, a polymerase comprises one or more active sites at which nucleotide binding and/or catalysis of nucleotide polymerization may occur. In some aspects, a polymerase includes other enzymatic activities, such as for example, 3′ to 5′ exonuclease activity or 5′ to 3′ exonuclease activity. In some aspects, a polymerase has strand displacing activity. A polymerase may include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some aspects, a polymerase may be isolated from a cell, or generated using recombinant DNA technology or chemical synthesis methods. In some aspects, a polymerase may be expressed in prokaryote, eukaryote, viral, or phage organisms. In some aspects, a polymerase may be post-translationally modified proteins or fragments thereof. A polymerase may be derived from a prokaryote, eukaryote, virus or phage. A polymerase comprises DNA-directed DNA polymerase and RNA-directed DNA polymerase.

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide may be complementary or non-complementary to a nucleotide residue in the template molecule. The nucleotide unit or the free nucleotide may bind to the 3′ end of the nucleic acid primer at a position that is opposite a complementary nucleotide residue in the nucleic acid template molecule. The persistence time is indicative of the stability of the binding complex and strength of the binding interactions. Persistence time may be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids may be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids comprise non-natural internucleoside linkages, including phosphorothioate, phosphorothiolate, or peptide nucleic acid (PNA) linkages. In some aspects, nucleic acids comprise a one type of polynucleotides or a mixture of two or more different types of polynucleotides.

The term “primer” and related terms used herein refers to an oligonucleotide, either natural or synthetic, that is capable of hybridizing with a DNA and/or RNA polynucleotide template to form a duplex molecule. Primers may have any length, but typically range from 4-50 nucleotides. A typical primer comprises a 5′ end and 3′ end. The 3′ end of the primer may include a 3′ OH moiety which serves as a nucleotide polymerization initiation site in a polymerase-mediated primer extension reaction. Alternatively, 3′ end of the primer may lack a 3′ OH moiety, or may include a terminal 3′ blocking group that inhibits nucleotide polymerization in a polymerase-mediated reaction. Any one nucleotide, or more than one nucleotide, along the length of the primer may be labeled with a detectable reporter moiety. A primer may be in solution (e.g., a soluble primer) or may be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid may be single-stranded or double-stranded, or the template nucleic acid may have single-stranded or double-stranded portions. The sequence of the template nucleic acid may be partially or wholly complementary to the sequence of the complementary strand. The template nucleic acid may be obtained from a naturally-occurring source, recombinant form, or chemically synthesized to include any type of nucleic acid analog. The template nucleic acid may be linear, circular, or other forms. The template nucleic acids may include an insert region having an insert sequence which is also known as a sequence of interest. The template nucleic acids may also include at least one adaptor sequence. The template nucleic acid may be a concatemer having two or tandem copies of a sequence of interest and at least one adaptor sequence. The insert region may be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region may be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region may be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid may be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization may comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing may be the standard A-T or C-G base pairing, or may be other forms of base-pairing interactions. Duplex nucleic acids may include mismatched base-paired nucleotides.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some aspects comprises a monophosphate, diphosphate, or triphosphate, or corresponding phosphate analog. In some aspects, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases may be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100:4319-48), acyclic moieties (Martinez, et al., 1999 Nucleic Acids Research 27:1271-1274; Martinez, et al., 1997 Bioorganic & Medicinal Chemistry Letters vol. 7:3013-3016), and other sugar moieties (Joeng, et al., 1993 J. Med. Chem. 36:2627-2638; Kim, et al., 1993 J. Med. Chem. 36:30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some aspects, nucleotides comprise a chain of one, two or three phosphorus atoms where the chain is typically attached to the 5′ carbon of the sugar moiety via an ester or phosphoramide linkage. In some aspects, the nucleotide is an analog having a phosphorus chain in which the phosphorus atoms are linked together with intervening O, S, NH, methylene or ethylene. In some aspects, the phosphorus atoms in the chain include substituted side groups including O, S or BH₃. In some aspects, the chain includes phosphate groups substituted with analogs including phosphoramidate, phosphorothioate, phosphordithioate, and O-methylphosphoroamidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation may be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties may be selected having spectrally distinct emission profiles, or having minimal overlapping spectral emission profiles. Reporter moieties may be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene) penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene) penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene) hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene) hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some aspects, the reporter moiety may be a FRET pair, such that multiple classifications may be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure may include but are not limited to: nucleotide transient-binding; nucleotide incorporation; de-blocking; washing; removing; flowing; detecting; imaging and/or identifying. Such linkage may comprise, for example, covalent, ionic, hydrogen, dipole-dipole, hydrophilic, hydrophobic, or affinity bonding, bonds or associations involving van der Waals forces, mechanical bonding, and the like. In some aspects, such linkage occurs intramolecularly, for example linking together the ends of a single-stranded or double-stranded linear nucleic acid molecule to form a circular molecule. In some aspects, such linkage may occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages may be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components may be linked together covalently. For example, two nucleic acid components may be enzymatically ligated together where the linkage that joins together the two components comprises phosphodiester linkage. A first and second nucleic acid component may be linked together, where the first nucleic acid component may confer a function on a second nucleic acid component. For example, linkage between a primer binding sequence and a sequence of interest forms a nucleic acid library molecule having a portion that may bind to a primer. In another example, a transgene (e.g., a nucleic acid encoding a polypeptide or a nucleic acid sequence of interest) may be ligated to a vector where the linkage permits expression or functioning of the transgene sequence contained in the vector. In some aspects, a transgene is operably linked to a host cell regulatory sequence (e.g., a promoter sequence) that affects expression of the transgene. In some aspects, the vector comprises at least one host cell regulatory sequence, including a promoter sequence, enhancer, transcription and/or translation initiation sequence, transcription and/or translation termination sequence, polypeptide secretion signal sequences, and the like. In some aspects, the host cell regulatory sequence controls expression of the level, timing and/or location of the transgene.

The term “adaptor” and related terms refers to oligonucleotides that may be operably linked (appended) to a target polynucleotide, where the adaptor confers a function to the co-joined adaptor-target molecule. Adaptors comprise DNA, RNA, chimeric DNA/RNA, or analogs thereof. Adaptors may include at least one ribonucleoside residue. Adaptors may be single-stranded, double-stranded, or have single-stranded and/or double-stranded portions. Adaptors may be configured to be linear, stem-looped, hairpin, or Y-shaped forms. Adaptors may be any length, including 4-100 nucleotides or longer. Adaptors may have blunt ends, overhang ends, or a combination of both. Overhang ends include 5′ overhang and 3′ overhang ends. The 5′ end of a single-stranded adaptor, or one strand of a double-stranded adaptor, may have a 5′ phosphate group or lack a 5′ phosphate group. Adaptors may include a 5′ tail that does not hybridize to a target polynucleotide (e.g., tailed adaptor), or adaptors may be non-tailed. An adaptor may include a sequence that is complementary to at least a portion of a primer, such as an amplification primer, a sequencing primer, or a capture primer (e.g., soluble or immobilized capture primers). Adaptors may include a random sequence or degenerate sequence. Adaptors may include at least one inosine residue. Adaptors may include at least one phosphorothioate, phosphorothiolate and/or phosphoramidate linkage. Adaptors may include a barcode sequence which may be used to distinguish polynucleotides (e.g., insert sequences) from different sample sources in a multiplex assay. Adaptors may include a unique identification sequence (e.g., unique molecular index, UMI; or a unique molecular tag) that may be used to uniquely identify a nucleic acid molecule to which the adaptor is appended. In some aspects, a unique identification sequence may be used to increase error correction and accuracy, reduce the rate of false-positive variant calls and/or increase sensitivity of variant detection. Adaptors may include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence may be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

In some aspects, the support is solid, semi-solid, or a combination of both. In some aspects, the support is porous, semi-porous, non-porous, or any combination of porosity. In some aspects, the support may be substantially planar, concave, convex, or any combination thereof. In some aspects, the support may be cylindrical, for example comprising a capillary or interior surface of a capillary.

In some aspects, the surface of the support may be substantially smooth. In some aspects, the support may be regularly or irregularly textured, including bumps, etched, pores, three-dimensional scaffolds, or any combination thereof.

In some aspects, the support comprises a bead having any shape, including spherical, hemi-spherical, cylindrical, barrel-shaped, toroidal, disc-shaped, rod-like, conical, triangular, cubical, polygonal, tubular or wire-like.

The support may be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some aspects, the surface of the support is coated with one or more compounds to produce a passivated layer on the support. In some aspects, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some aspects, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some aspects, a static contact angle may be determined. In some aspects, an advancing or receding contact angle may be determined. In some aspects, the water contact angle for the hydrophilic, low-binding support surfaced disclosed herein may range from about 0 degrees to about 30 degrees. In some aspects, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

Aspects of the present disclosure provide a plurality (e.g., two or more) of nucleic acid templates immobilized to a support. In some aspects, the immobilized plurality of nucleic acid templates have the same sequence or have different sequences. In some aspects, individual nucleic acid template molecules in the plurality of nucleic acid templates are immobilized to a different site on the support. In some aspects, two or more individual nucleic acid template molecules in the plurality of nucleic acid templates are immobilized to a site on the support. In some aspects, the support comprises a plurality of sites arranged in an array. The term “array” refers to a support comprising a plurality of sites located at pre-determined locations on the support to form an array of sites. The sites may be discrete and separated by interstitial regions. In some aspects, the pre-determined sites on the support may be arranged in one dimension in a row or a column, or arranged in two dimensions in rows and columns. In some aspects, the plurality of pre-determined sites is arranged on the support in an organized fashion. In some aspects, the plurality of pre-determined sites is arranged in any organized pattern, including rectilinear, hexagonal patterns, grid patterns, patterns having reflective symmetry, patterns having rotational symmetry, or the like. The pitch between different pairs of sites may be that same or may vary. In some aspects, the support may have nucleic acid template molecules immobilized at a plurality of sites at a surface density of about 10²-10¹⁵ sites per mm², or more, to form a nucleic acid template array. In some aspects, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some aspects, a plurality of pre-determined sites on the support (e.g., 10²-10¹⁵ sites or more) are immobilized with nucleic acid templates to form a nucleic acid template array. In some aspects, the nucleic acid templates that are immobilized at a plurality of pre-determined sites by hybridization to immobilized surface capture primers, or the nucleic acid templates are covalently attached to the surface capture primers. In some aspects, the nucleic acid templates that are immobilized at a plurality of pre-determined sites, for example immobilized at 10²-10¹⁵ sites or more. In some aspects, the nucleic acid templates that are immobilized at a plurality of sites on the support comprise linear or circular nucleic acid template molecules or a mixture of both linear and circular molecules. In some aspects, the immobilized nucleic acid templates are clonally-amplified to generate immobilized nucleic acid polonies at the plurality of pre-determined sites. In some aspects, individual immobilized nucleic acid template molecules comprise one copy of a target sequence of interest, or comprise concatemers having two or more tandem copies of a target sequence of interest.

In some aspects, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some aspects, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some aspects, a plurality of randomly located sites on the support (e.g., 10²-10¹⁵ sites or more) are immobilized with nucleic acid templates to form a support immobilized with nucleic acid templates. In some aspects, the nucleic acid templates that are immobilized at a plurality of randomly located sites by hybridization to immobilized surface capture primers, or the nucleic acid templates are covalently attached to the surface capture primer. In some aspects, the nucleic acid templates that are immobilized at a plurality of randomly located sites, for example immobilized at 10²-10¹⁵ sites or more. In some aspects, the nucleic acid templates that are immobilized at a plurality of sites on the support comprise linear or circular nucleic acid template molecules or a mixture of both linear and circular molecules. In some aspects, the immobilized nucleic acid templates are clonally-amplified to generate immobilized nucleic acid polonies at the plurality of randomly located sites. In some aspects, individual immobilized nucleic acid template molecules comprise one copy of a target sequence of interest, or comprise concatemers having two or more tandem copies of a target sequence of interest.

In some aspects, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations and/or buffers and the like) onto the support so that the plurality of immobilized nucleic acid template molecules on the support may be reacted with the reagents in a massively parallel manner. In some aspects, the fluid communication of the plurality of immobilized nucleic acid template molecules may be used to conduct nucleotide binding assays and/or conduct nucleotide polymerization reactions (e.g., primer extension or sequencing) on the plurality of immobilized nucleic acid template molecules, and to conduct detection and imaging for massively parallel sequencing. In some aspects, the term “immobilized” and related terms refer to nucleic acid molecules or enzymes (e.g., polymerases) that are attached to the support at pre-determined or random locations, where the nucleic acid molecules or enzymes are attached directly to a support through covalent bond or non-covalent interaction, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some aspects, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some aspects, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some aspects, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some aspects, the number of layers is at least 1, at least 2, at least 3, at least 4, at least 5, at least 6, at least 7, at least 8, at least 9, or at least 10. In some aspects, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some aspects the number of layers may range from about 2 to about 4. In some aspects, all of the layers may comprise the same material. In some aspects, each layer may comprise a different material. In some aspects, the plurality of layers may comprise a plurality of materials. In some aspects at least one layer may comprise a branched polymer. In some aspect, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some aspects the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some aspects, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some aspects, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group may be either on the side chain of the polymer or directly attaches to a central core or central backbone of the polymer. The branched polymer may have linear backbone with one or more functional groups coming off the backbone for conjugation. The branched polymer may also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and it variants refers to a nucleic acid template molecule that has been subjected to one or more amplification reactions either in-solution or on-support. In the case of in-solution amplified template molecules, the resulting amplicons are distributed onto the support. Prior to amplification, the template molecule comprises a sequence of interest and at least one universal adaptor sequence. In some aspects, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

As used herein, the term “sequencing” and its variants comprise obtaining sequence information from a nucleic acid strand, typically by determining the identity of at least some nucleotides (including their nucleobase components) within the nucleic acid template molecule. While in some aspects, “sequencing” a given region of a nucleic acid molecule includes identifying each and every nucleotide within the region that is sequenced, in some aspects “sequencing” comprises methods whereby the identity of only some of the nucleotides in the region is determined, while the identity of some nucleotides remains undetermined or incorrectly determined. Any suitable method of sequencing may be used. In an exemplary aspect, sequencing may include label-free or ion based sequencing methods. In some aspects, sequencing may include labeled or dye-containing nucleotide or fluorescent based nucleotide sequencing methods. In some aspects, sequencing may include polony-based sequencing or bridge sequencing methods. In some aspects, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299 (5607): 682-686; Eid, et al., 2009 Science 323 (5910): 133-138; U.S. Pat. Nos. 7,170,050; 7,302,146; and 7,405,281). An example of sequence-by-hybridization includes SOLID sequencing (e.g., from Life Technologies; WO 2006/084132). An example of sequence-by-binding includes Omniome sequencing (e.g., U.S. Pat. No. 10,246,744).

While preferred aspects of the present invention have been shown and described herein, it will be obvious to those skilled in the art that such aspects are provided by way of example only. The breadth and scope of this disclosure should not be limited by any of the above-described exemplary aspects. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

1. A computer-implemented method for predicting quality of base calling in sequencing, comprising:
selecting, by a processor, a plurality of predictors;
determining, by the processor, a corresponding value for each of the plurality of predictors from one or more flow cell images; and
determining, by the processor, a quality score from a look-up table based on the corresponding value for each of the plurality of predictors, wherein the look-up table comprises a plurality of dimensions corresponding to the plurality of predictors and is generated based on a training data set.

2. The computer-implemented method of claim 1, wherein one or more of the plurality of predictors are indicative of an image quality of the one or more flow cell images.

3. The computer-implemented method of claim 1 further comprising:
acquiring, by a sequencing system, the one or more flow cell images from multiple channels of one or more samples immobilized on a flow cell; and
performing, by the processor, one or more preprocessing steps to generate the one or more flow cell images from multiple channels, the one or more preprocessing steps comprising:
(1) background subtraction;
(2) color correction;
(3) phasing or prephasing correction; and
(4) and normalization.

4-7. (canceled)

8. The computer-implemented method of claim 1 further comprising:
ranking, by the processor, candidate predictors based on corresponding effects thereof on quality scores of base calling, and
wherein selecting, by the processor, the plurality of predictors is based on the ranking of the candidate predictors.

9. (canceled)

10. The computer-implemented method of claim 8, wherein the plurality of predictors comprises: a first predictor of clarity, a second predictor of max_intensity, and a third predictor of low intensity median clarity.

11. (canceled)

12. The computer-implemented method of claim 1, wherein the plurality of predictors comprises a first predictor of clarity, a second predictor of max_intensity, and a third predictor of low intensity median clarity, and a fourth predictor of phasing and prephasing.

13. The computer-implemented method of claim 12, wherein the first predictor of clarity comprises a ratio of a max_intensity to a second max_intensity of a polony in a selected imaging cycle.

14. The computer-implemented method of claim 12, wherein the second predictor of max_intensity comprises an intensity from a brightest channel among four channels of a polony in a selected imaging cycle.

15. (canceled)

16. The computer-implemented method of claim 13, wherein the max_intensity comprises a normalized intensity of a brightest channel among four channels.

17. The computer-implemented method of claim 13, wherein the second max_intensity comprises an intensity from a second brightest channel among different channels of a polony in a selected imaging cycle.

18. The computer-implemented method of claim 13, wherein the second max_intensity comprises an intensity from a second brightest channel among 4 channels of a polony in a selected imaging cycle.

19. (canceled)

20. The computer-implemented method of claim 12, wherein the third predictor of low intensity median clarity is indicative of signal density of polonies within a region in a selected imaging cycle in the one or more flow cell images.

21. The computer-implemented method of claim 12, wherein the third predictor of low intensity median clarity is inversely proportional to the signal intensity in the one or more flow cell images.

22. The computer-implemented method of claim 12, wherein the third predictor of low intensity median clarity comprises a median clarity of the signal intensity of dim polonies in a region of a selected imaging cycle of the one or more flow cell images.

23. (canceled)

24. (canceled)

25. The computer-implemented method of claim 12, wherein the third predictor of low intensity median clarity is a median value of an inverse of clarity of selected dim polonies.

26. The computer-implemented method of claim 12, wherein the corresponding value of the first predictor of clarity is based on a polony of signals in the one or more flow cell images, the one or more flow cell images being within an imaging cycle.

27. The computer-implemented method of claim 12, wherein the corresponding value of the second predictor of max_intensity is based on the polony of signals in the one or more flow cell images, the one or more flow cell images being within an imaging cycle.

28. The computer-implemented method of claim 12, wherein the corresponding value of the third predictor of low intensity median clarity is based on a region with multiple polonies in the one or more flow cell images, the one or more flow cell images being within an imaging cycle.

29. The computer-implemented method of claim 12, wherein the corresponding value of the fourth predictor of phasing and prephasing is based on multiple polonies in the one or more flow cell images, the one or more flow cell images being within an imaging cycle.

30-55. (canceled)

56. A computer-implemented method for predicting quality of base calling in sequencing, comprising:
obtaining, by a processor, correct base calls and erroneous bases calls by comparing training base calls to reference base calls;
dividing, by the processor, a corresponding range of value for each of a plurality of predictors in training regions into a corresponding number of bins;
initializing, by the processor, a look-up table having a number of dimensions determined by the plurality of predictors and each dimension comprising the corresponding number of bins;
determining, by the processor, a first number of correct base calls and a second number of erroneous base calls in each bin of the look-up table; and
iterating, by the processor and until the first number of correct base calls and the second number of erroneous base calls in each bin are no greater than a predetermined number, one or more of the following steps:
(a) calculating a standard quality score and a conservative quality score for each bin of the look-up table;
(b) determining coordinates of a bin with a maximum conservative quality score in the look-up table;
(c) assigning a selected number of bins with the standard quality score that corresponds to the determined coordinates in the look-up table; and
(d) setting correct base calls and erroneous base calls in the selected number of bins to be a predetermined number.

57-94. (canceled)

95. A computer-implemented system for predicting quality of base calling in sequencing, comprising:
one or more hardware processors;
one or more data storage devices storing instructions executable by the one or more hardware processors to cause the one or more hardware processors to perform operations, the operations comprising:
selecting a plurality of predictors;
determining a corresponding value for each of the plurality of predictors from one or more flow cell images; and
determining a quality score from a look-up table based on the corresponding value for each of the plurality of predictors, wherein the look-up table comprises a plurality of dimensions corresponding to the plurality of predictors and is generated based on a training data set.

96-102. (canceled)

103. The computer-implemented method of claim 1, wherein the determining the quality score occurs prior to performing base calling of one or more samples based on the one or more flow cell images.
